(19) United States
(12) Patent Application Publication
(10) Pub. No.: US 2025/0199009 A1
(43) Pub. Date: Jun. 19, 2025

(54) CLICK-CHEMISTRY FUNCTIONALIZED COMPOUNDS AND METHODS OF CONJUGATION

(71) Applicant: AAT Bioquest, Inc., Pleasanton, CA (US)

(72) Inventors: Zhenjun DIWU, Danville, CA (US); Jianjun HE, Pleasanton, CA (US); Jixiang LIU, Pleasanton, CA (US)

(21) Appl. No.: 18/389,590

(22) Filed: Dec. 19, 2023

(51) Int. Cl. G01N 33/68; C07D 235/14; C07D 271/10; C07D 403/14; C07D 413/12; C07D 413/14; C07D 495/04; C07J 43/00; G01N 21/64

(52) U.S. Cl. CPC G01N 33/68; C07D 235/14; C07D 271/10; C07D 403/14; C07D 413/12; C07D 413/14; C07D 495/04; C07J 43/003; G01N 21/6428; G01N 2021/6439

(57) ABSTRACT

The disclosure relates in general to click-chemistry functionalized compounds, biological conjugates and conjugation methods for use in biological assays.

### BACKGROUND

Click chemistry was developed by K. Barry Sharpless as a robust and specific method of ligating two molecules together. It has been widely used in drug discovery, biomolecular conjugations, and material science (see U.S. Pat. No. 8,802,852 to Gessner et al; U.S. Pat. No. 9,176,380 to Bowman et al; U.S. Pat. No. 10,550,385 to Zewge et al; U.S. Pat. No. 10,648,985 to Kyle et al; U.S. Pat. No. 11,091,588 to Yang et al; U.S. Pat. No. 11,519,027 to Sharp et al). In general, click chemistry reactions proceed with high selectivity and good yield. Two important characteristics make click chemistry so attractive for assembling compounds, reagents, and biomolecules for preclinical and clinical applications. First, click reactions are bio-orthogonal, thus the click chemistry-functionalized biomolecules would not react with the natural biomolecules that lack a clickable functional group. Second, the reactions proceed with ease under mild conditions, such as at room temperature and in aqueous media.

Among all the click reactions, the copper-catalyzed azide-alkyne [3+2] dipolar cycloaddition (CuAAC) to produce a triazole is the most common one. These reactions can be used to modify one cellular component while leaving others unharmed or untouched. Click chemistry has found increasing applications in all aspects of drug discovery in medicinal chemistry, such as for generating lead compounds through combinatorial methods. Bioconjugation via click chemistry is rigorously employed in proteomics and nucleic research. In radiochemistry, selective radiolabeling of biomolecules in cells and living organisms for imaging and therapy has been realized by this technology. Bifunctional chelating agents for several radionuclides useful for positron emission tomography and single-photon emission computed tomography imaging have also been prepared by using click chemistry (see Stuyver et al., U.S. Pat. No. 7,919,247; Kyle et al., U.S. Pat. No. Zhang, et al., U.S. Pat. No. 10,648,985; Bertozzi et al., U.S. Pat. No. 9,410,958; Front. Chem., 2021, 17, 1, www.frontiersin.org/articles/10.3389/fchem.2021.774977/full; Kolb et al, Angew. Chem. Int. Ed, 2001, 40, 2004, onlinelibrary.wiley.com/doi/epdf/10.1002/1521-3773%2820010601%2940%3A11%3C2004%3A%3AAID-ANIE2004%3E3.0.CO%3B2-5; Nwe and Brechbier, Cancer Biother Radiopharm. 2009, 24, 289-302, 10.1089/cbr.2008.0626). The classic click reactions typically require both Cu(I) ion and a Cu(I) chelator to proceed efficiently. The Cu(I)-chelating ligands, such as THPTA or BTTAA, are generally used to stabilize the Cu(I) oxidation state in aqueous solution. However, it is well-known that Cu(I) ion is toxic to cells and can have deleterious effects on cells and other biological samples. The Cu(I)-chelating ligands may have other undesired side effects too. Therefore, the reduction or complete elimination of Cu(I) ion and its chelator used in click reactions would have significant benefits for drug discovery and conjugating biomolecules.

### SUMMARY

The compounds of the disclosure contain a copper-chelating ligand that significantly stabilizes the Cu(I) oxidation state and thus accelerates the copper-catalyzed azide-alkyne cycloaddition reaction. The compounds of the disclosure do not require the use of external copper-chelators (such as the common THPTA or BTTAA) that are usually used at high concentration. The high concentration of copper chelators is known to cause biocompatibility in biological samples. The introduction of a copper-chelating moiety at the reporter molecule allows for a dramatic raise of the effective Cu(I) concentration at the reaction site and thus accelerates the reaction. The azides and alkynes of the disclosure can form strong, active copper complexes that may serve as both reactant and catalyst in the click reaction. Under extremely mild conditions, the compounds of the disclosure react much faster in high yield compared to the existing CuAAC reactions performed with a conventional azide or alkyne. The unprecedented reactivity of the click compounds is of special value for detecting low abundance targets, improving biocompatibility, and being used for any other demanding applications.

### BRIEF DESCRIPTION OF THE DRAWINGS

The foregoing and other features and advantages of the present embodiments will be more fully understood from the following detailed description of illustrative embodiments taken in conjunction with the accompanying drawings in which:

FIG. 1 shows the reaction scheme of an azido compound of the disclosure with a biological substrate that is modified with an alkyne compound. The click reaction can occur under extremely mild conditions (e.g., [Azide]=0.02 mM, [Alkyne]=0.02 mM, [CuSO₄]=0.02 mM, [Sodium Ascorbate]=0.5 mM, in 100 mM HEPES) under which a common azido compound may not effectively react with an alkyne-modified substrate. The azido compounds of the disclosure contain a copper-chelating ligand that significantly stabilizes the Cu(I) oxidation state in aqueous solution and accelerates the copper-catalyzed azide-alkyne cycloaddition (CuAAC) reaction.

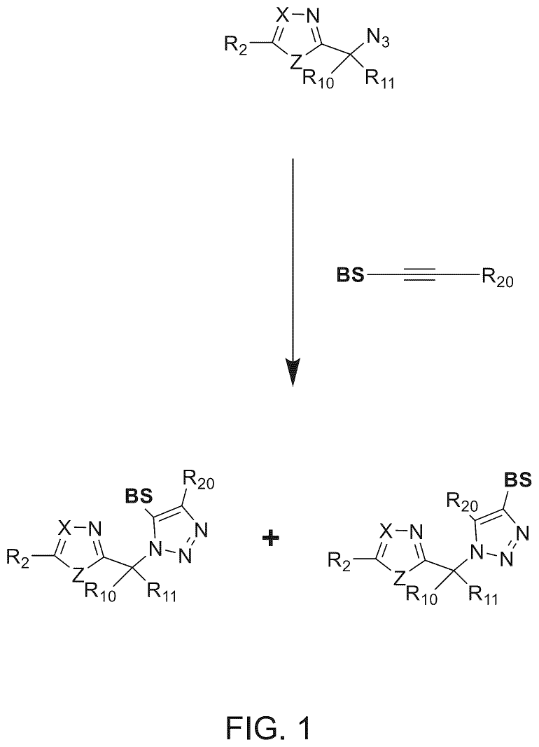

FIG. 2 shows the reaction scheme of an alkyne compound of the disclosure with a biological substrate that is modified with an azido compound. The click reaction can occur under extremely mild conditions (e.g., [Azide]=0.02 mM, [Alkyne]=0.02 mM, [CuSO₄]=0.02 mM, [Sodium Ascorbate]=0.5 mM, in 100 mM HEPES) under which a common alkyne compound may not effectively react with an azido-modified substrate. The alkyne compounds of the disclosure contain a copper-chelating ligand that significantly stabilizes the Cu(I) oxidation state in aqueous solution and accelerates the copper-catalyzed azide-alkyne cycloaddition (CuAAC) reaction.

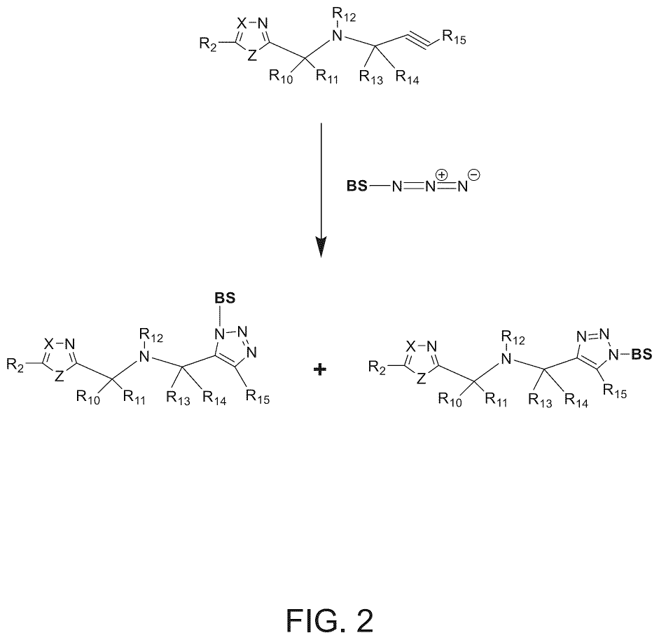

FIG. 3 shows the comparative measurements for the click reactions are performed in 100 mM HEPES in which [Azide]=0.2 mM, [Alkyne]=0.2 mM, [CuSO₄]=0.2 mM, [Sodium Ascorbate]=5 mM. Compound 16 is a representative copper-chelating azide of the disclosure while Cy5 azide (commercially available from AAT Bioquest) is a representative conventional azide (used as the reference standard). Both Compound 16 and Cy5 azide contain the same Cy5 fluorophore. The click reaction is run as described in Example 24. As seen from FIG. 3, in the absence of an external chelator (such as THPTA), Compound 16 (see Reaction B) allows the formation of azide copper complexes that react almost instantaneously with coumarin alkyne under mild conditions. Under the same reaction conditions, there is very little reactivity with Cy5 azide (see Reaction A). It is apparent that Compound 16 is a much more reactive substrate while Cy5 azide demonstrates very little reactivity. In conclusion, the azides of the disclosure react much faster in high yield with an alkyne compared to the CuAAC reaction performed with a conventional azide.

FIG. 4 shows the comparative measurements for the click reactions are performed in 100 mM HEPES in which [Azide]=0.02 mM, [Alkyne]=0.02 mM, [CuSO₄]=0.02 mM, [Sodium Ascorbate]=0.5 mM. Compound 16 is a representative copper-chelating azide of the disclosure while Cy5 azide (commercially available from AAT Bioquest) is a representative conventional azide (used as the reference standard). Both Compound 16 and Cy5 alkyne contain the same Cy5 fluorophore. The click reaction is run as described in Example 25. As seen from FIG. 4, in the absence of an external chelator (such as THPTA), Compound 16 (see Reaction B) allows the formation of azide copper complexes that react with coumarin alkyne under extremely mild conditions. Under the same conditions, there is no reaction detected with Cy5 azide (see Reaction A). It is apparent that Compound 16 is a much more reactive substrate while Cy5 azide demonstrates no reactivity. In conclusion, the azides of the disclosure react much faster in high yield with an alkyne compared to the CuAAC reaction performed with a conventional azide.

FIG. 5 shows the comparative measurements for the click reactions are performed in 100 mM HEPES in which [Azide]=0.2 mM, [Alkyne]=0.2 mM, [CuSO₄]=0.2 mM, [Sodium Ascorbate]=5 mM. Compound 21 is a representative copper-chelating alkyne of the disclosure while Cy5 alkyne (commercially available from AAT Bioquest) is a representative conventional alkyne (used as the reference standard). Both Compound 21 and Cy5 alkyne contain the same Cy5 fluorophore. The click reaction is run as described in Example 26. As seen from FIG. 5, in the absence of an external chelator (such as THPTA), Compound 21 (see Reaction B) allows the formation of alkyne copper complexes that react almost instantaneously with AMCA azide under mild conditions. Under the same reaction conditions, there is very little reactivity with Cy5 alkyne (see Reaction A). It is apparent that Compound 21 is a much more reactive substrate while Cy5 alkyne demonstrates very little reactivity. In conclusion, the alkynes of the disclosure react much faster in high yield with an alkyne compared to the CuAAC reaction performed with a conventional alkyne.

FIG. 6 shows the comparative measurements for the click reactions are performed in 100 mM HEPES in which [Azide]=0.02 mM, [Alkyne]=0.02 mM, [CuSO₄]=0.02 mM, [Sodium Ascorbate]=0.5 mM. Compound 21 is a representative copper-chelating alkyne of the disclosure while Cy5 alkyne (commercially available from AAT Bioquest) is a representative conventional alkyne (used as the reference standard). Both Compound 21 and Cy5 alkyne contain the same Cy5 fluorophore. The click reaction is run as described in Example 27. As seen from FIG. 6, in the absence of an external chelator (such as THPTA), Compound 21 (see Reaction B) allows the formation of alkyne copper complexes that react almost instantaneously with AMCA azide under mild conditions. Under the same reaction conditions, there is no reaction detected with Cy5 alkyne (see Reaction A). It is apparent that Compound 21 is a much more reactive substrate while Cy5 alkyne demonstrates no reactivity. In conclusion, the alkynes of the disclosure react much faster in high yield with an alkyne compared to the CuAAC reaction performed with a conventional alkyne.

FIG. 7 (panels A and B) show the S-phase HeLa cells are labelled and imaged with Compound 16 in combination with 5-ethynyl uridine. The cells are treated with 5-ethynyl uridine as described in Example 29. The alkyne-modified cells are reacted with Compound 16 and imaged with a Keyence fluorescence microscope using a Cy5 filter set. (A). Control without 5-ethynyl uridine added; (B). With 5-ethynyl uridine added.

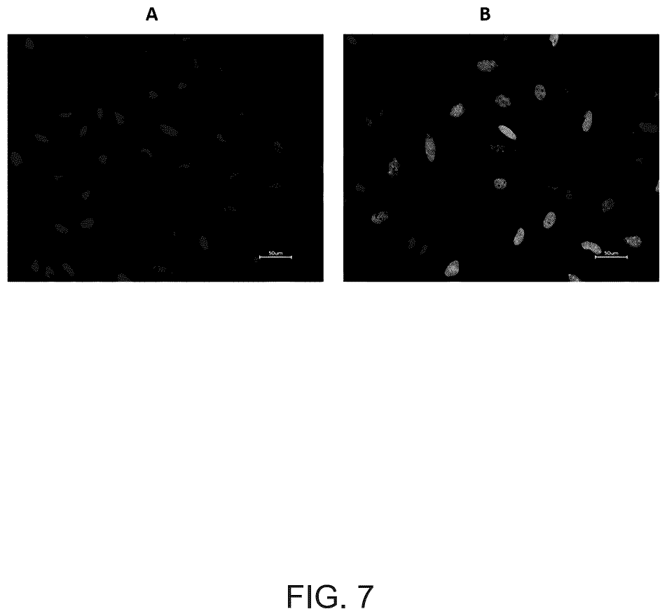

### DETAILED DESCRIPTION

When describing the embodiments of the present disclosure, which may include compounds and pharmaceutically acceptable salts thereof, pharmaceutical compositions containing such compounds and methods of using such compounds and compositions, the following terms, if present, have the following meanings unless otherwise indicated

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The abbreviations used herein have their conventional meaning without the chemical and biological arts. The chemical structures and formulae set forth herein are constructed according to the standard rules of chemical valency known in the chemical arts.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl”, “cycloaliphatic”, or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms. In some embodiments, aliphatic groups contain 1-2 aliphatic carbon atoms. In some embodiments, “cycloaliphatic” (or “carbocyclyl” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substituted nitrogen of a heterocyclic ring, for example N (as in 3,4-dihydro-2H-pyrrolyl), NH (as in pyrrolidinyl) or NR⁺ (as in N-substituted pyrrolidinyl)).

The term “heteroaliphatic” refers to an aliphatic moiety that contains at least one heteroatom in the chain, for example, an amine, carbonyl, carboxy, oxo, thio, phosphate, phosphonate, nitrogen, phosphorus, silicon, or boron atoms in place of a carbon atom. In one embodiment, the only heteroatom is nitrogen. In one embodiment, the only heteroatom is oxygen. In one embodiment, the only heteroatom is sulfur. “Heteroaliphatic” is intended herein to include, but is not limited to, heteroalkyl, heteroalkenyl, heteroalkynyl, heterocycloalkyl, heterocycloalkenyl, and heterocycloalkynyl moieties. In one embodiment, “heteroaliphatic” is used to indicate a heteroaliphatic group (cyclic, acyclic, substituted, unsubstituted, branched or unbranched) having 1-20 carbon atoms. In one embodiment, the heteroaliphatic group is optionally substituted in a manner that results in the formation of a stable moiety. Nonlimiting examples of heteroaliphatic moieties are polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

The term “unsaturated”, as used herein, means that a moiety has one or more units of unsaturation.

Before the present disclosure is described in further detail, it is to be understood that this disclosure is not limited to the particular molecules, methodologies, devices, solutions or apparatuses described, as such methods, devices, solutions or apparatuses can, of course, vary. It is also to be understood that the terminology used herein is for the purpose of describing particular embodiments only, and is not intended to limit the scope of the present disclosure. The following definitions are set forth to illustrate and define the meaning and scope of the various terms used to describe the disclosure herein.

Use of the singular forms “a”, “an”, and “the” include plural references unless the context clearly dictates otherwise. Thus, for example, reference to “a probe” includes a plurality of probes, and the like. Additionally, use of specific plural references, such as “two” “three” etc., read on larger numbers of the same subject unless the context clearly dictates otherwise.

Terms such as “connected”, “attached”, “conjugated”, and “linked” are used interchangeably herein and encompass direct as well as indirect connection, attachment, linkage or conjugation unless the context clearly dictates otherwise.

The term “alkyl” as used herein, by itself or as part of another group, refers to straight, branched chain or cyclic radicals having up to 50 carbons, unless the chain length or ring size is limited thereto, such as methyl, ethyl, propyl, cyclopropanyl, isopropyl, butyl, t-butyl, isobutyl, pentyl, hexyl, cyclohexyl, isohexyl, heptyl, 4,4-dimethylpentyl, octyl, 2,2,4-trimethylpentyl, nonyl, and decyl, among others.

The term “alkylene” as employed herein, by itself or as part of another group, refers to straight, branched chain or cyclic divalent radicals having up to 50 carbons, unless the chain length or ring size is limited thereto. Typical examples include methylene (—CH₂—), ethylene (—CH₂CH₂—), propylene, butylene, pentylene, hexylene, heptylene, octylene, nonylene, and decylene, among others.

The term “alkenyl” as used herein, by itself or as part of another group, means a straight, branched chain or cyclic radical having 2-50 carbon atoms and one or more carbon-carbon double bonds, unless the chain length or ring size is limited thereto, such as ethenyl, 1-propenyl, 2-propenyl, 2-methyl-1-propenyl, 1-butenyl, and 2-butenyl, among others. The alkenyl chain may be 2 to 10 carbon atoms in length. Alternatively, the alkenyl chain may be 2 to 4 carbon atoms in length.

The term “alkenylene” as used herein, by itself or as part of another group, means straight, branched chain or cyclic divalent radical having 2-50 carbon atoms, unless the chain length or ring size is limited thereto, said straight, branched chain or cyclic radical containing at least one carbon-carbon double bond. Typical examples include ethenylene (—CH═CH—), propenylene (—CH═CHCH₂— and —CH₂CH═CH—), n-butenylene, and 3-methyl-2-pentenylene, hexenylene, heptenylene, octenylene, nonenylene, and decenylene, among others.

The term “alkynyl” as used herein, by itself or as part of another group, means a straight, branched chain or cyclic radical of 2-50 carbon atoms, unless the chain length or ring size is limited thereto, having at least one carbon-carbon triple bond between two of the carbon atoms in the chain, such as acetylenyl, 1-propynyl, and 2-propynyl, among others. The alkynyl chain may be 2 to 10 carbon atoms in length. Alternatively, the alkynyl chain may be from 2 to 4 carbon atoms in length.

The term “alkynylene” as used herein, by itself or as part of another group, means a straight, branched chain or cyclic divalent radical having 2-50 carbon atoms, unless the chain length or ring size is limited thereto, that contains at least one carbon-carbon triple bond. Typical examples include ethynylene (—C≡C—), propynylene (—C≡CCH₂— and —CH₂C≡C—), n-butynylene, 4-methyl-2-pentynylene, 1-butynylene, 2-butynylene, 3-butynylene, 4-butynylene, pentynylene, hexynylene, heptynylene, octynylene, nonynylene, and decynylene, among others.

The term “alkoxy” as used herein, by itself or as part of another group, refers to any of the above radicals linked via an oxygen atom. Typical examples include methoxy, ethoxy, isopropyloxy, sec-butyloxy, n-butyloxy, t-butyloxy, n-pentyloxy, 2-methylbutyloxy, 3-methylbutyloxy, n-hexyloxy, and 2-ethylbutyloxy, among others. Alkoxy also may include PEG groups (—OCH₂CH₂O—) or alkyl moieties that contain more than one oxygen atom.

The term “aryl” as employed herein, by itself or as part of another group, refers to an aryl or aromatic ring system containing 1 to 10 unsaturated rings (each ring containing 6 conjugated carbon atoms and no heteroatoms) that are optionally fused to each other or bonded to each other by carbon-carbon single bonds, that is optionally further substituted as described below. Examples of aryl ring systems include, but are not limited to, substituted or unsubstituted derivatives of fluorenyl, phenyl, biphenyl, o-, m-, or p-terphenyl, 1-naphthyl, 2-naphthyl, 1-, 2-, or 9-anthryl, 1-, 2-, 3-, 4-, or 9-phenanthrenyl, 1-perylenyl, 1-ovalenyl, 1-benzoperyenyl, 1- or 2-chrysenyl, 1- or 2-hexahelicenyl, 1-corannulenyl, 1-coronenyl, 1-, 2- or 4-pyrenyl. Aryl substituents may include phenyl, substituted phenyl, naphthyl or substituted naphthyl.

The term “heteroaryl” as employed herein, by itself or as part of another group, refers to groups having 5 to 14 ring atoms; 6, 10 or 14 π electrons shared in a cyclic array; and containing carbon atoms and 1, 2, 3, or 4 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, carbazolyl, phenanthridinyl, acridinyl, perimidinyl, phenanthrolinyl, phenazinyl, isothiazolyl, phenothiazinyl, isoxazolyl, furazanyl, phenoxazinyl, and tetrazolyl groups.

Any aryl or heteroaryl ring system is unsubstituted or optionally and independently substituted by any synthetically accessible and chemically stable combination of substituents, such as H, halogen, cyano, sulfo, alkali, or ammonium salt of sulfo, nitro, carboxy, alkyl, perfluoroalkyl, alkoxy, alkylthio, amino, monoalkylamino, dialkylamino or alkylamido, the alkyl portions of which having 18 or fewer carbons.

The term “biological detection moiety” or “BDM” as employed herein, by itself or as part of another group, refers to moiety that can be biologically monitored or detected, e.g., biotin, DNP, Digoxigenin (DIG), FLAG, HIS, AU1, AU5, Myc, T7, VSV-G, E-Tag, S-Tag, Avi, HSV, MBP, CBP, GST, K3, TK15, beta-Gal, enzymes, antibodies, lectins, glycoproteins, histones, albumins, lipoproteins, avidin, streptavidin, protein A, protein G, GFP, phycobiliproteins and other fluorescent proteins, hormones, toxins, chemokines and growth factors etc.

The term “fluorescent dye” or “FD” as employed herein, by itself or as part of another group, refers to an aromatic or heteroaromatic moiety that emits fluorescence longer than 500 nm. The examples of fluorescent dyes include, but not limited to coumarins, fluoresceins, rhodamines, cyanines, bodipys, phthalocyanines, porphyrins, acridines, acridones, DDAO, carbazines, anthrancences, anthraquinones, DRAQ-5, azulenes, benzoxazinones, azacoumarins, benzoimidazoles, benzoxazoles, benzothiazoles, tetrapyrroles, diketopyrrolopyrrole, pyrazolines, hypericins, hypocrellins, perylenequinones, IR-140, luciferin, naphthamides, naphthalenes, naphthoquinones, NBD, SBD, oxazines, oxazoles, dapoxyls, osmium complexes, ruthenium complexes, platinum complexes, polycyclic dyes, pyrilium salts, nanocrystals, rhodoles, Schiff bases, squraines, styryls, polythiophenes, tetrazolium salts or upconversion oxides.

The terms “halogen” or “halo” as employed herein, by itself or as part of another group, refers to chlorine, bromine, fluorine or iodine.

The term “carboxy” as used herein, by itself or as part of another group, is represented by —COOW wherein W is a hydrogen, an alkali metal ion, an ammonium or other biologically compatible counter ion.

The term “sulfonate” as used herein, by itself or as part of another group, is represented by —S(═O)₂OW wherein W is a hydrogen, an alkali metal ion, an ammonium or other biologically compatible counter ion.

The term “phosphonate” as used herein, by itself or as part of another group, is represented by —P(═O)O₂W₂ wherein W is a hydrogen, an alkali metal ion, an ammonium or other biologically compatible counter ion.

The term “boronate” as used herein, by itself or as part of another group, is represented by —B(OW)₂ wherein W is a hydrogen, an alkali metal ion, an ammonium or other biologically compatible counter ion.

The term “ammonium” as used herein, by itself or as part of another group, is represented —N(R₃)X wherein n is 1-20, R is a short alkyl (e.g. C₁-C₁₂ alkyl); X is a biologically compatible anion such as F⁻, Cl⁻, Br⁻ or I⁻. Ammonium may include a nitrogen ring structure such as pyridinium, acridinium or quinolinium etc.

The term “sulfonium” as used herein, by itself or as part of another group, is represented —S(R₂)X wherein n is 1-20, R is a short alkyl (e.g. C₁-C₁₂ alkyl);

X is a biologically compatible anion such as F⁻, Cl⁻, Br⁻ or I⁻.

The term “phosphonium” as used herein, by itself or as part of another group, is represented —P(R₃)X wherein n is 1-20, R is a short alkyl (e.g. C₁-C₁₂ alkyl);

X is a biologically compatible anion such as F⁻, Cl⁻, Br⁻ or I⁻.

The term “polyethyleneglycol” or “PEG” as used herein, by itself or as part of another group, is represented by —(OCH₂CH₂O)ₙ— wherein n is 2-30.

The term “water soluble group” or “WSG” as used herein, by itself or as part of another group, is a moiety or substituent capable of increasing the water solubility of a compound or moiety to which it is attached or incorporated, e.g., relative to a compound or moiety lacking the WSG. WSGs of interest include, but are not limited to PEG, carboxy, sulfonate, phosphonate, boronate, amine, ammonium, sulfonium, phosphonium, alcohol or hydroxy, or sugar.

The terms “functional group” or “FG” as used herein, by itself or as part of another group, is a reactive moiety that can be used to covalently link compounds of the disclosure to a biological target. In some embodiments, the FG is a chemoselective group. In some embodiments, the FG is a click chemistry group. FG's of interest include, but are not limited to activated esters, acrylamides, acyl azides, acyl halides, acyl nitriles, aldehydes, ketones, alkyl halides. Alkyl sulfonates, anhydrides, aryl halides, aziridines, boronates, carbodiimides, diazoalkanes, epoxides, haloacetamides, haloplatinate, halotriazines, imido esters, isocyanates, isothiocyanates, maleimides, phosphoramidites, silyl halides, sulfonate esters, sulfonyl halides, 1,2,4,5-tetrazines, hydroxylamines, hydrazines, cysteines, nitrile-N-oxides, anthracenes, amines, anilines, thiols, alcohols, phenols, carboxylic acids, glycols, heterocycles, alkynes, cyclooctynes, or methyl 2-diphenylphosphinobenzonate.

The term “linker or L” as used herein, by itself or as part of another group, is a spacer that links a compound of this disclosure to a reactive moiety or a biological target. They include, but are not limited to alkyl, alkyaryl, alkylheteroaryl, aryl, heteroaryl or a PEG.

The term “conjugated compound” as used herein, by itself or as part of another compound, is a compound containing an extended series of randomly interconnected unsaturated bonds, aryls and/or heteroaryls.

The term “biological substrate” or “BS” as used herein, by itself or as part of another group, is a biological target molecule. They include, but are not limited to antibodies, antigens, proteins, peptides, oligonucleotides, DNA, RNA, PNA, aptamers, sugars, antibiotics, metabolites, cAMP, cGMP, polysaccharides, viruses, cells and tissues. The term “L-BS” refers to a linked biological substrate or molecule, e.g., that is connected to another moiety via a linker.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As used herein and unless otherwise specified, the suffix “-ene” is used to describe a bivalent group. Thus, any of the terms above can be modified with the suffix “-ene” to describe a bivalent version of that moiety. For example, a bivalent carbocycle is “carbocyclylene”, a bivalent aryl ring is “arylene”, a bivalent benzene ring is “phenylene”, a bivalent heterocycle is “heterocyclylene”, a bivalent heteroaryl ring is “heteroarylene”, a bivalent alkyl chain is “alkylene”, a bivalent alkenyl chain is “alkylene”, a bivalent alkynyl chain is “alkynylene”, and so forth.

As described herein, compounds of the disclosure may, when specified, contain “optionally substituted” moieties. In general, the term “substituted”, whether preceded by the term “optionally” or not, means that one or more hydrogens of the designated moiety are replaced with a suitable substituent. “Substituted” applies to one or more hydroens that are either explicit or implicit from the structure

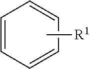

refers to at least

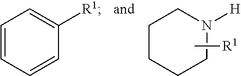

refers to at least

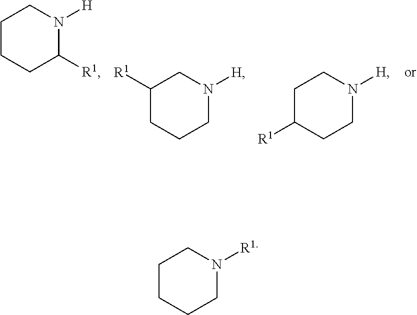

In addition, in a polycyclic ring system, substituents may, unless otherwise indicated, replace a hydrogen on any individual ring

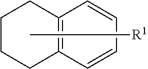

refers to at least

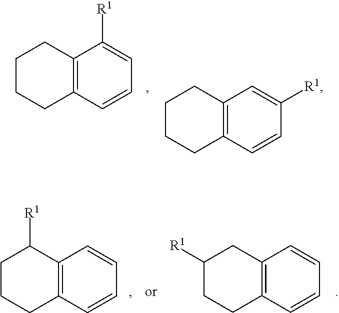

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their purification, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}; —O(CH₂)₀₋₄C(O)OR^{◯}; —O(CH₂)₀₋₄OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph, which may be substituted with R^{◯}, —CH═CHPh, which may be substituted with R^{◯}; —(CH₂)₀₋₄(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)N(R^{◯})₂; —N(R^{◯})C(S)N(R^{◯})₂; —(CH₂)₀₋₄N(R^{◯})C(S)N(R^{◯})₂; —(CH₂)₀₋₄N(R^{◯})C(O)OR^{◯}; —N(R^{◯})N(R^{◯})C(O)R^{◯}; —N(R^{◯})N(R^{◯})C(O)N(R^{◯})₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)Osi(R^{◯})₃; —(CH₂)₀₋₄OC(O)R^{◯}; —OC(O)(CH₂)₀₋₄SR^{◯}; —SC(S)SR^{◯}; —(CH₂)₀₋₄SC(O)R^{◯}; —(CH₂)₀₋₄C(O)N(R^{◯})₂; —C(S)N(R^{◯})₂; —C(S)SR^{◯}; —SC(S)SR^{◯}; —(CH₂)₀₋₄OC(O)N(R^{◯})₂; —C(O)N(OR^{◯})R^{◯}; —C(O)C(O)R^{◯}; —C(O)CH_{2C}(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}; —(CH₂)₀₋₄S(O)R^{◯}; —N(R^{◯})S(O)₂N(R^{◯})₂; —N(R^{◯})S(O)₂R^{◯}; —N(OR^{◯})R^{◯}; —C(NH)N(R^{◯})₂; —P(OR^{◯})₂; —P(O)(R^{◯})₂, —OP(O)(R^{◯})₂, —OP(O)(OR^{◯})₂; —SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5- to 6-membered heteroaryl ring), or a 5- to 6-membered saturated, partially unsaturated, or aryl ring having 0-4 heteroatoms independently selected from nitrogen, oxygen, and sulfur, or, notwithstanding the definition above, two independent occurrences of R^{◯}, taken together with their intervening atoms(s), form a 3- to 12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{◯} (or the ring formed by taking two independent occurrences of R^{◯} together with their intervening atoms), are independently halogen; —(CH₂)₀₋₂R^{●}; -(haloR^{●}), —(CH₂)₀₋₂OH; —(CH₂)₀₋₂OR^{●}; —(CH₂)₀₋₂CH(OR^{●})₂; —O(haloR^{●}); —CN; —N₃; —(CH₂)₀₋₂C(O)R^{●}; —(CH₂)₀₋₂C(O)OH; —(CH₂)₀₋₂C(O)OR^{●}; —(CH₂)₀₋₂SR^{●}; —(CH₂)₀₋₂SH; —(CH₂)₀₋₂NH₂; —(CH₂)₀₋₂NHR^{●}; —(CH₂)₀₋₂NR^{●}₂; —NO₂, —SiR^{●}₃; -OsiR^{●}₃; —C(O)SR^{●}; —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur. Suitable divalent substituents on a saturated carbon atom of R^{◯} include ═O and ═S.

Suitable divalent substituents on a saturated carbon atom of an “optionally substituted” group include the following: ═O; ═S; ═NNR^{#}₂; ═NNHC(O)R^{#}₂; ═NNHC(O)OR^{#}₂; ═NNHS(O)₂R^{#}₂; ═NR^{#}; ═NOR^{#}; —O(C(R^{#}₂))₂₋₃O—; or —S(C(R^{#}₂))₂₋₃S—; wherein each independent occurrence of R^{#} is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR^{#}₂)₂₋₃O—, wherein each independent occurrence of R^{#} is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, and sulfur.

Suitable substituents on the aliphatic group of R^{#} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}₂; wherein each R^{†} is independently hydrogen, C1-6 aliphatic which may be substituted as defined below, unsubstituted —Oph, or an unsubstituted 5- to 6-membered saturated, partially unsaturated, or aryl ring having 0-4 heteroatoms independently selected from nitrogen, oxygen, and sulfur, or, notwithstanding the definition above, two independent occurrences or R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

### Compounds

In some embodiments, provided herein is a compound, wherein the compound is represented by Formula A or is a salt thereof:
wherein

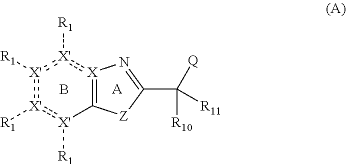

X is CR₁ or N;
Z is O, S, N—R₈, P—R₈, O═P—R₈, or O═P—OR₈;
Q is N₃, N(R₁)₂

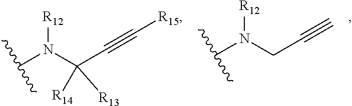

alkynyl or substituted alkynyl;
each X′ is independently absent or X,
   wherein, when present 1 to 4 X′ together form Ring B, fused to Ring A;
each of R₁ and R₂ is independently selected from H, halogen, or an optionally substituted FG, BDM, L, L-BS, aliphatic, heteroaliphatic, allyl, allenyl, propargyl, carboxy, sulfonate, PEG, WSG, alkoxy, aryloxy, alkylthiol, arylthiol, nitro, nitroso, cyano, amino, hydroxy, phosphonyl, sulfonyl, carbonyl, boronic acid, aryl, and heteroaryl;
each of R₈, R₁₀, and R₁₁ is independently selected from H or an optionally substituted alkyl, heteroalkyl, FG, BDM, L-BS, acyl, sulfonyl, aryl, and heteroaryl;
each of R₁₂, R₁₃, R₁₄, and R₁₅ is selected from independently H, alkyl, FG, BDM, L-FG, L-BDM, L, L-BS, FD, L-FD, acyl, sulfonyl, aryl, heteroaryl, and optionally substituted versions thereof.

In some embodiments, the compound of Formula A is represented by Formula I or is a salt thereof:

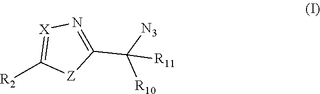

wherein
   X is CR₁ or N;
   Z is O, S, N—R₈, P—R₈, O═P—R₈, or O═P—OR₈;
   each of R₁ and R₂ is independently selected from H, halogen, or an optionally substituted FG, BDM, L-BS, aliphatic, heteroaliphatic, carboxy, sulfonate, PEG, WSG, alkoxy, aryloxy, alkylthiol, arylthiol, nitro, nitroso, cyano, amino, hydroxy, phosphonyl, sulfonyl, carbonyl, boronic acid, aryl, and heteroaryl;
   each of R₈, R₁₀, and R₁₁ is independently selected from H or an optionally substituted alkyl, FG, BDM, L-BS, acyl, sulfonyl, aryl, and heteroaryl;

In some embodiments, X is N. In some embodiments, Z is O, S, or N—R₈. In some embodiments, R₂ is selected from H, or an optionally substituted carboxy, sulfonate, PEG, alkoxy, aryloxy, aryl, and heteroaryl. In some embodiments, each of R₈, R₁₀ and R₁₁ is independently H or an optionally substituted alkyl, alkylamino, aryl, and heteroaryl.

In some embodiments,
X is N;
Z is O, S, or N—R₈;
R₁ or R₂ is selected from

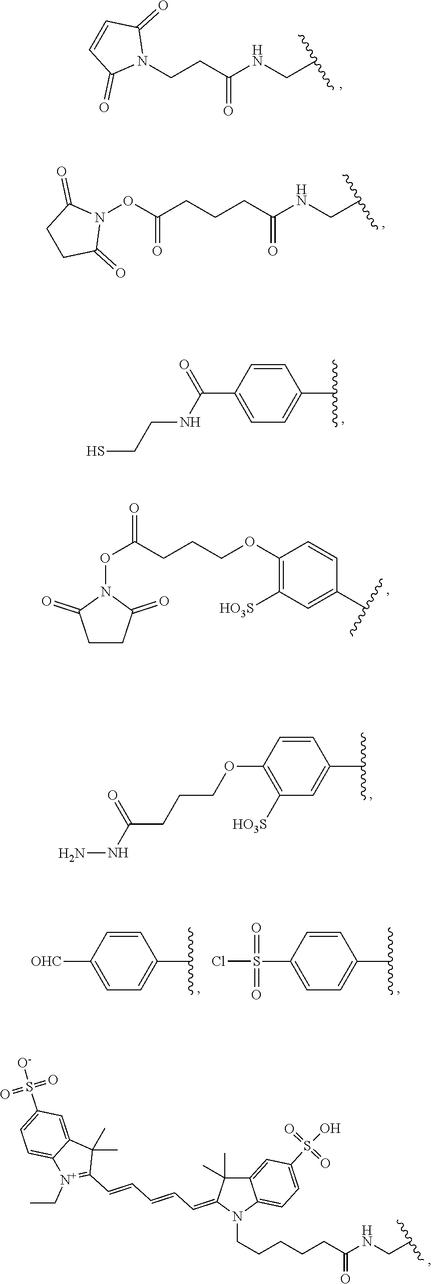

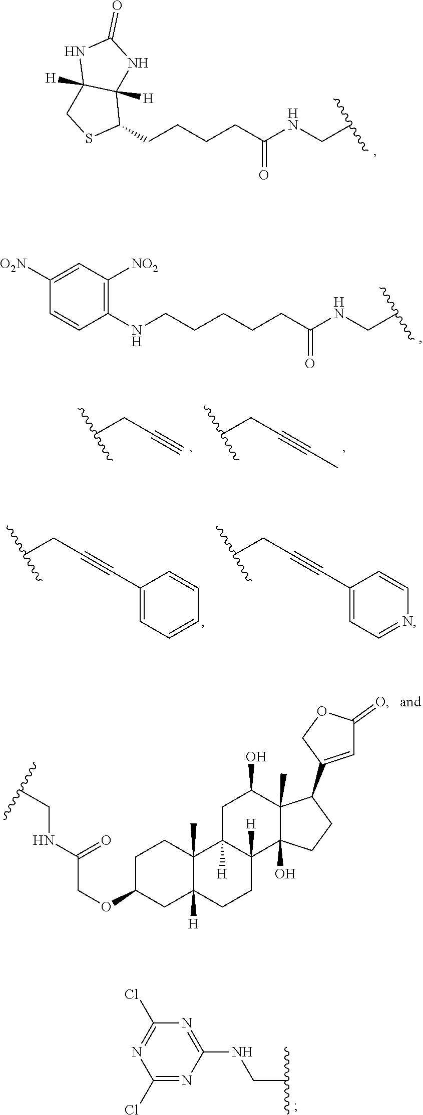

In some embodiments,
X is CR₁;
Z is O, S, or N—R₈;
each of R₁ and R₂ is independently selected from

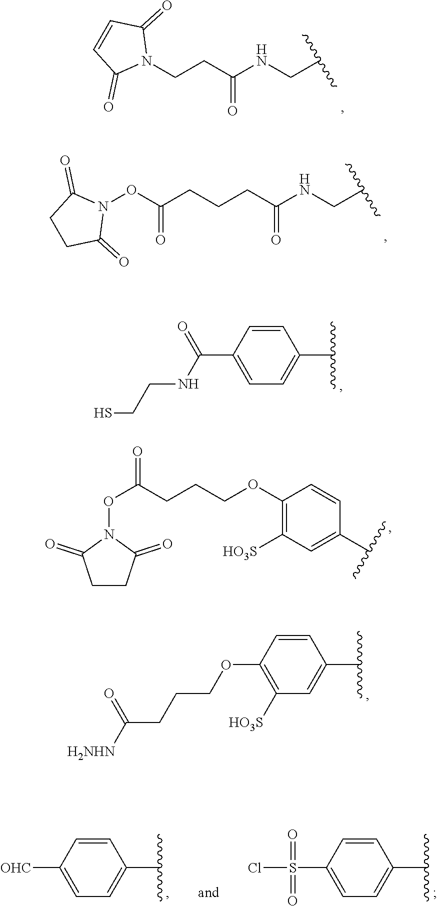

each of R₈, R₁₀, and R₁₁ is independently selected from H or an optionally substituted alkyl, aryl, and heteroaryl.

In some embodiments, at least one of R₁, R₂, R₈, R₁₀, or R₁₁ is or comprises FD.

In some embodiments, FD is selected from coumarin, styryl, fluorescein, rhodamine, cyanine, BODIPY, phthalocyanine, porphyrin, and fluorescent protein.

In some embodiments, the compound of Formula A is selected from

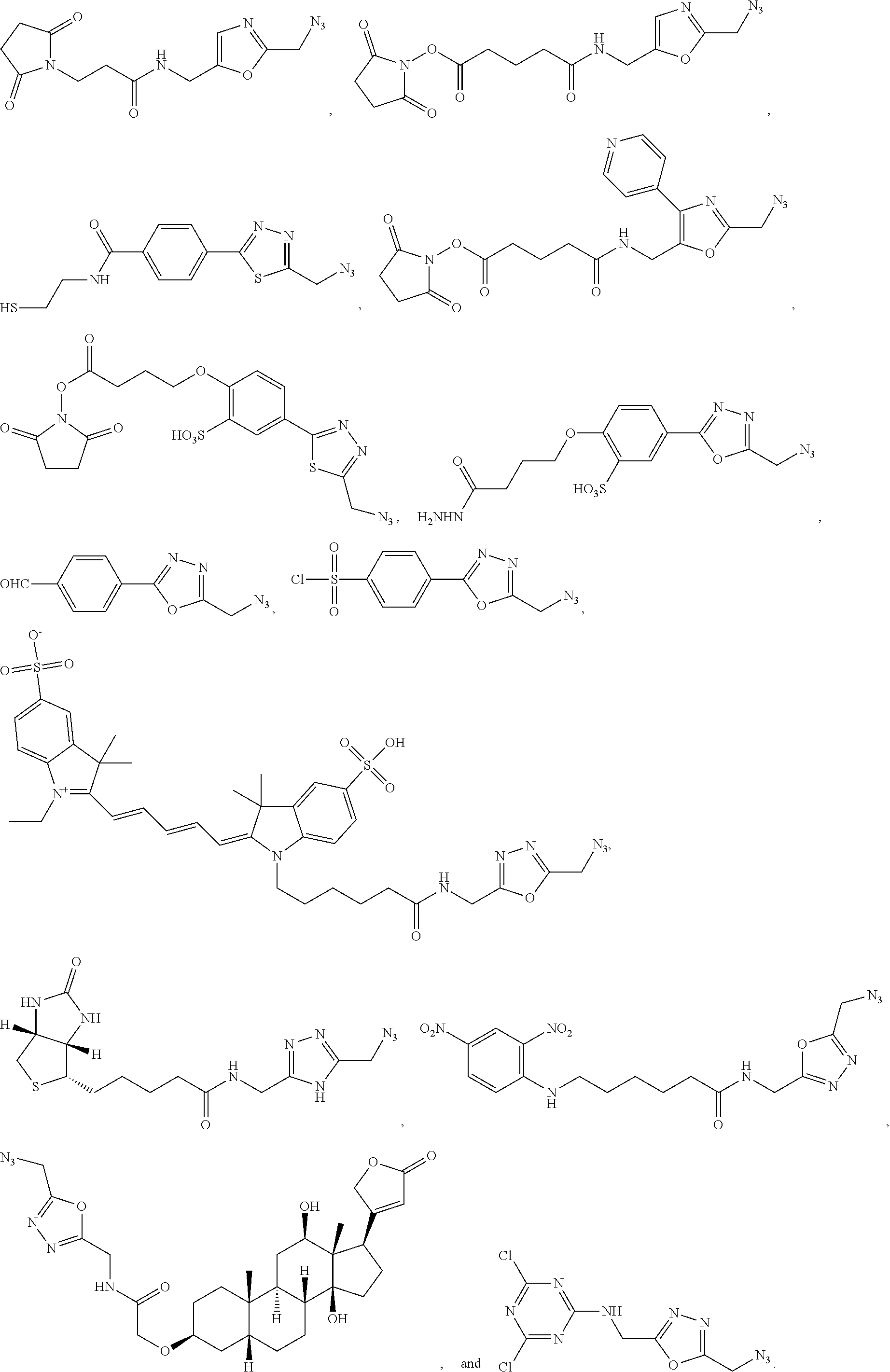

In some embodiments, the compound is represented by Formula II:

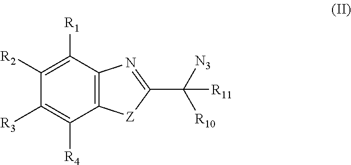

wherein
   Z is O, S, N—R₈, P—R₈, O═P—R₈, or O═P—OR₈;
   each of R₁, R₂, R₃, and R₄ is independently selected from H, halogen, or an optionally substituted FG, BDM, L-BS, carboxy, sulfonate, PEG, WSG, alkoxy, aryloxy, thiol, alkylthiol, arylthiol, nitro, nitroso, cyano, amino, hydroxy, phosphonyl, sulfonyl, carbonyl, boronic acid, aryl, or heteroaryl;
   each of R₈, R₁₀, and R₁₁ is independently selected from H or an optionally substituted alkyl, FG, BDM, L-BS, aryl, and heteroaryl.

In some embodiments,
Z is O, S, or N—R₈;
each of R₁, R₂, R₃, and R₄ is independently selected from H, halogen, or an optionally substituted carboxy, sulfonate, PEG, alkoxy, aryloxy, aryl, and heteroaryl;
each of R₈, R₁₀, and R₁₁ is independently selected from H or an optionally substituted alkyl, aryl, and heteroaryl.

In some embodiments, each of R₁, R₁, R₂, R₃, R₄, and R₈ is independently selected from H, halogen,

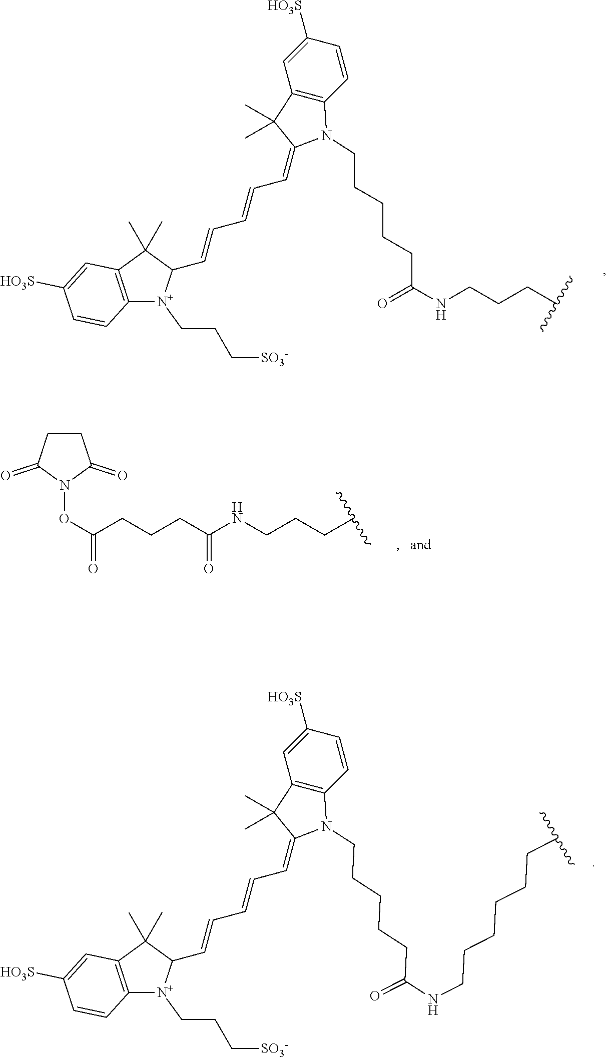

In some embodiments, WSG is selected from sulfonate, PEG, carboxyalkyl, sulfonylalkyl, phosphonylalkyl, hydroxyalkyl, aminoalkyl, and ammoniumylalkyl.

In some embodiments, FG is selected from an activated ester, aldehyde, maleimide, amino, hydroxylamine, and hydrazine.

In some embodiments, one of R₁ to R₁₁ comprises a FD.

In some embodiments, BS is an antibody, antigen, protein, peptide, oligonucleotide, DNA, RNA, PNA, aptamer, or a cell.

In some embodiments, each of R₁, R₂, R₃, R₄, and R₈ is independently selected from

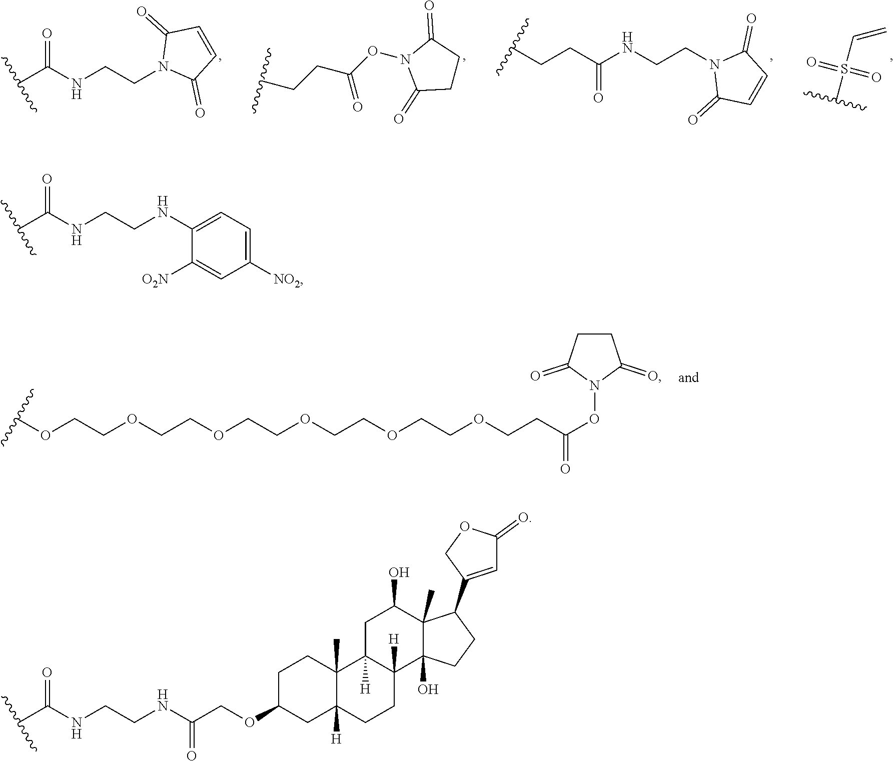

In some embodiments, the compound is selected from

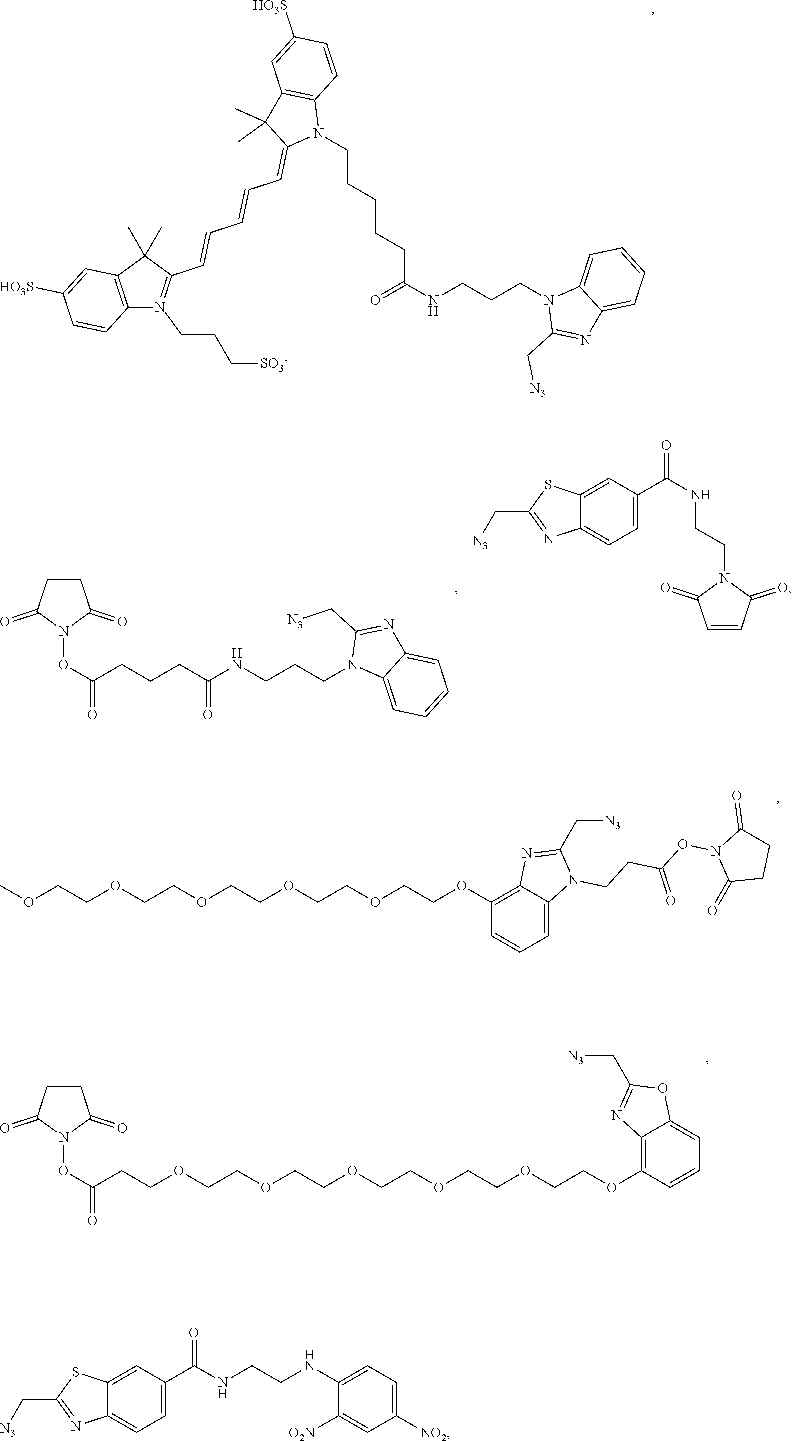

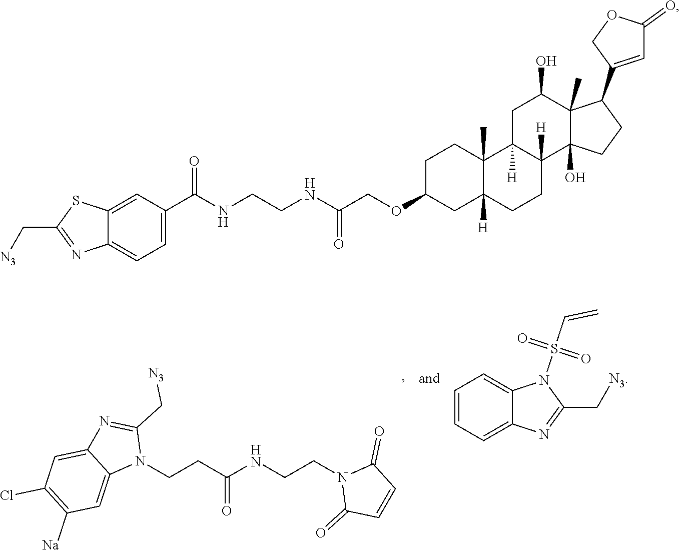

In some embodiments, the compound of Formula A is represented by Formula III:

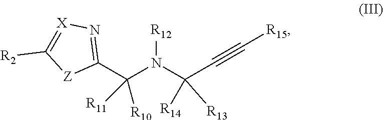

wherein
   X is CR₁ or N;
   Z is O, S, N—R₈, P—R₈, O═P—R₈, or O═P—OR₈;
   each of R₁ and R₂ is independently selected from H, halogen, or an optionally substituted FG, BDM, L-BS, carboxy, sulfonate, PEG, WSG, alkoxy, aryloxy, thiol, alkylthiol, arylthiol, nitro, nitroso, cyano, amino, hydroxy, phosphonyl, sulfonyl, carbonyl, boronic acid, aryl, or heteroaryl;
   each of R₈, R₁₀, R₁₁, R₁₂, R₁₃, R₁₄, and R₁₅ is selected from independently H or an optionally substituted alkyl, FG, BDM, L-BS, acyl, sulfonyl, aryl, and heteroaryl.

In some embodiments, X is CR₁;
Z is O, S, or N—R₈;
R₁ and R₂ are combined to form an aryl or heteroaryl;
each of R₈, R₁₀, and R₁₁ is independently selected from H or an optionally substituted alkyl, aryl, or heteroaryl.

In some embodiments, X is N;
Z is O, S, or N—R₈;
R₂ is H, halogen, carboxy, sulfonate, a PEG, alkoxy, aryloxy, aryl, or heteroaryl;
each of R₈, R₁₀, R₁₁, R₁₂, R₁₃, R₁₄, and R₁₅ is independently selected from H, or an optionally substituted alkyl, aryl, and heteroaryl.

In some embodiments, X is N;
Z is O, S, or N—R₈;
R₂ is H, halogen, carboxy, sulfonate, a PEG, alkoxy, aryloxy, aryl, or heteroaryl;
each of R₈, R₁₀, R₁₁, R₁₂, R₁₃ R₁₄, and R₁₅ is independently selected from H, or an optionally substituted alkyl, aryl, or heteroaryl;
R₁₂ is acyl or sulfonyl.

In some embodiments, R₁₅ is H, CH₃,

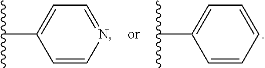 [SMILES: *c1ccncc1 ; *c1ccccc1]

In some embodiments, WSG is sulfonate, PEG, carboxyalkyl, sulfonylalkyl, phosphonylalkyl, hydroxyalkyl, aminoalkyl, or tetraalkylammonium.

In some embodiments, wherein FG is an activated ester, aldehyde, maleimide, amino, hydroxylamine, or hydrazine.

In some embodiments, at least one of R₁, R₂, R₈, R₁₀, R₁₁, R₁₂, R₁₃, R₁₄, and R₁₅ comprises a FD.

In some embodiments, R₁₂ is independently selected from

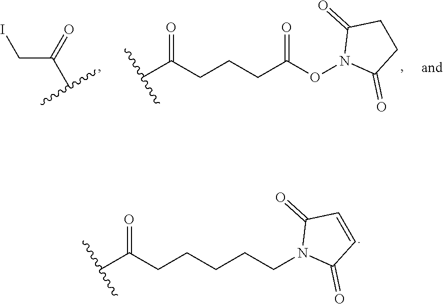 [SMILES: *C(=O)CI ; *C(=O)CCCC(=O)ON1C(=O)CCC1=O ; *C(=O)CCCCCN1C(=O)C=CC1=O]

In some embodiments, BS is an antibody, antigen, protein, peptide, oligonucleotide, DNA, RNA, PNA, aptamer, or cell.

In some embodiments, the compound is selected from

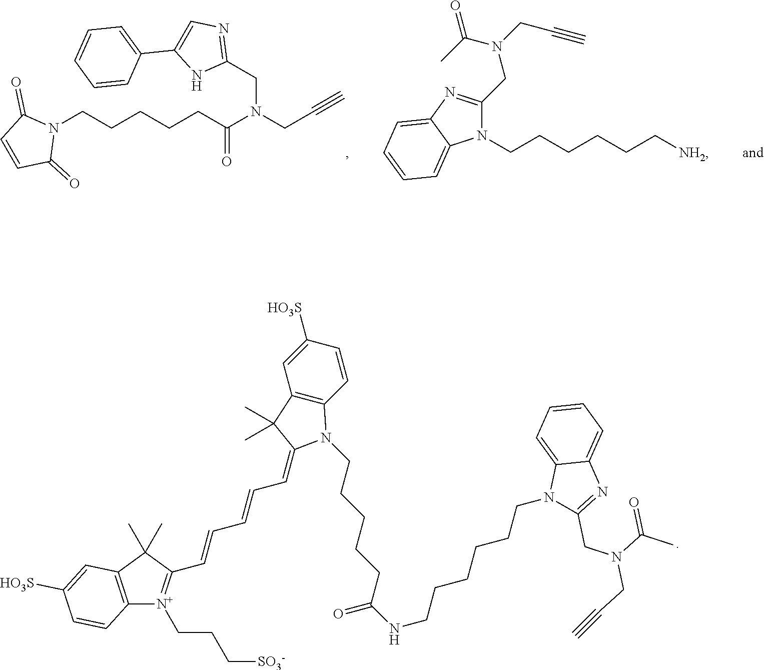

In some embodiments, the compound of Formula A is represented by Formula IV:

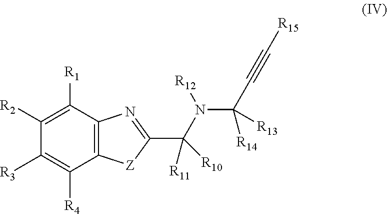

wherein
   Z is O, S, N—R₈, P—R₈, O═P—R₈, or O═P—OR₈;
   each of R₁, R₂, R₃, and R₄ is independently selected from H, halogen, or an optionally substituted FG, BDM, L-BS, carboxy, sulfonate, PEG, WSG, alkoxy, aryloxy, thiol, alkylthiol, arylthiol, nitro, nitroso, cyano, amino, hydroxy, phosphonyl, sulfonyl, carbonyl, boronic acid, aryl, and heteroaryl;
   each of R₈, R₁₀, R₁₁, R₁₂, R₁₃, R₁₄, R₁₅ is independently selected from H, or an optionally substituted alkyl, FG, BDM, L-BS, acyl, sulfonyl, aryl, and heteroaryl.

In some embodiments,
Z is O, S, or N—R₈;
each of R₁, R₂, R₃, and R₄ is independently selected from H, halogen, or an optionally substituted carboxy, sulfonate, PEG, alkoxy, aryloxy, aryl, and heteroaryl;
each of R₈, R₁₀, R₁₁, R₁₂, R₁₃, R₁₄, and R₁₅ is independently selected from H, or an optionally substituted alkyl, aryl, and heteroaryl.

In some embodiments,
Z is O, S, or N—R₈;
R₂ is H, halogen, or an optionally substituted carboxy, sulfonate, PEG, alkoxy, aryloxy, aryl, or heteroaryl;
each of R₈, R₁₀, R₁₁, R₁₃, R₁₄ and R₁₅ is independently selected from H or an optionally substituted alkyl, aryl, and heteroaryl;
R₁₂ is an acyl or sulfonyl.

In some embodiments, R₁₅ is H, CH₃,

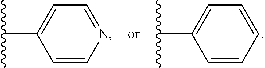 [SMILES: *c1ccncc1 ; *c1ccccc1]

In some embodiments, WSG is a sulfonate, PEG, carboxyalkyl, sulfonylalkyl, phosphonylalkyl, hydroxyalkyl, aminoalkyl, or trialkylammonium.

In some embodiments, FG is selected from an activated ester, aldehyde, maleimide, amino, hydroxylamine, and hydrazine.

In some embodiments, at least one of R₁ to R₁₅ contains a FD.

In some embodiments, each of R₁, R₂, R₃, R₄, and R₈ is independently selected from

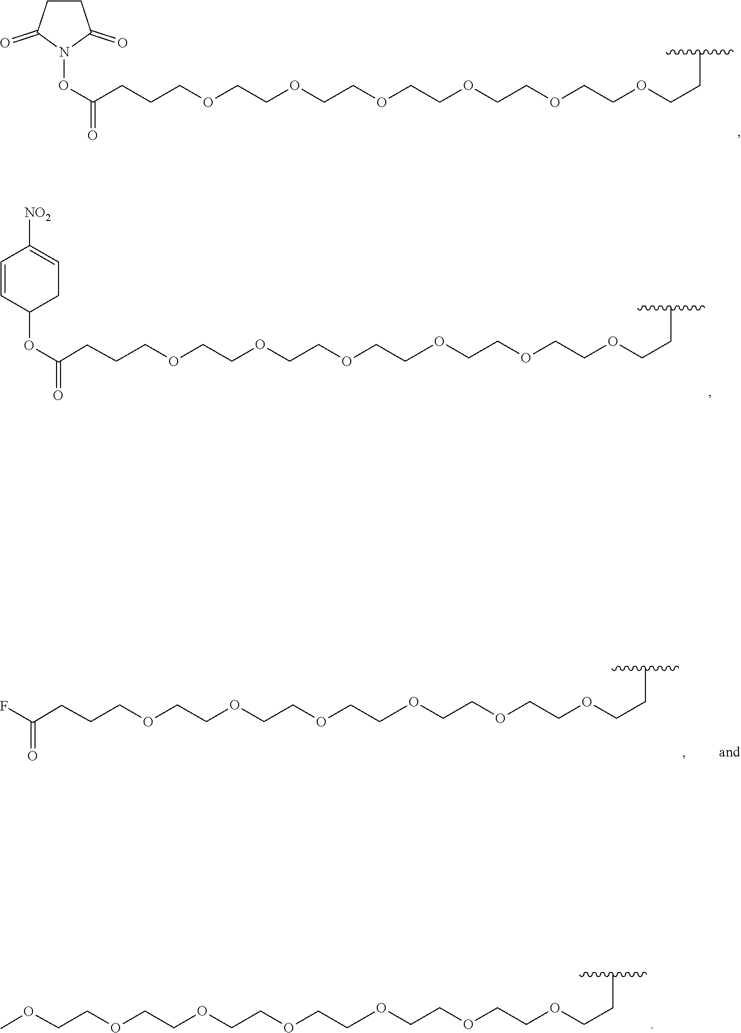 [SMILES: *CCOCCOCCOCCOCCOCCOCCCC(=O)ON1C(=O)CCC1=O ; *CCOCCOCCOCCCOCCOCCOCCCC(=O)OC1C=CC([N+](=O)[O-])=CC1 ; *CCOCCOCCOCCCOCCOCCOCCCC(=O)F ; *CCOCCOCCOCCCOCCOCCOCCOC]

In some embodiments, R₁₂ is independently selected from

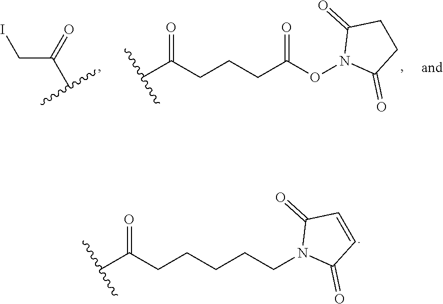 [SMILES: *C(=O)CI ; *C(=O)CCCC(=O)ON1C(=O)CCC1=O ; *C(=O)CCCCCN1C(=O)C=CC1=O]

In some embodiments, BS is an antibody, an antigen, a protein, a peptide, an oligonucleotide, a DNA, an RNA, a PNA, an aptamer or a cell.

In some embodiments, the compound is selected from

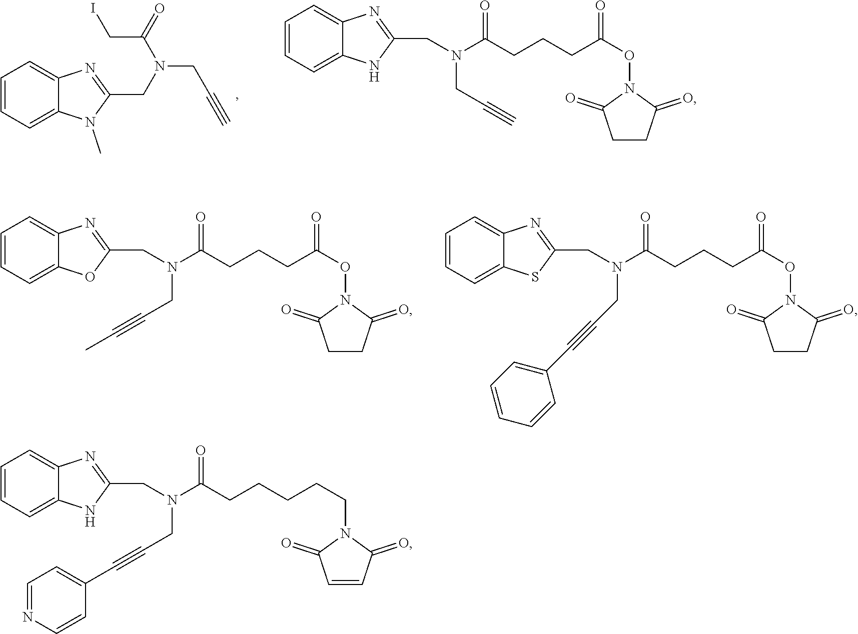 [SMILES: C#CCN(Cc1nc2ccccc2n1C)C(=O)CI ; "," ; C#CCN(Cc1nc2ccccc2[nH]1)C(=O)CCCC(=O)ON1C(=O)CCC1=O ; CC#CCN(Cc1nc2ccccc2o1)C(=O)CCCC(=O)ON1C(=O)CCC1=O ; O=C(CCCC(=O)N(CC#Cc1ccccc1)Cc1nc2ccccc2s1)ON1C(=O)CCC1=O ; O=C(CCCCCN1C(=O)C=CC1=O)N(CC#Cc1ccncc1)Cc1nc2ccccc2[nH]1]

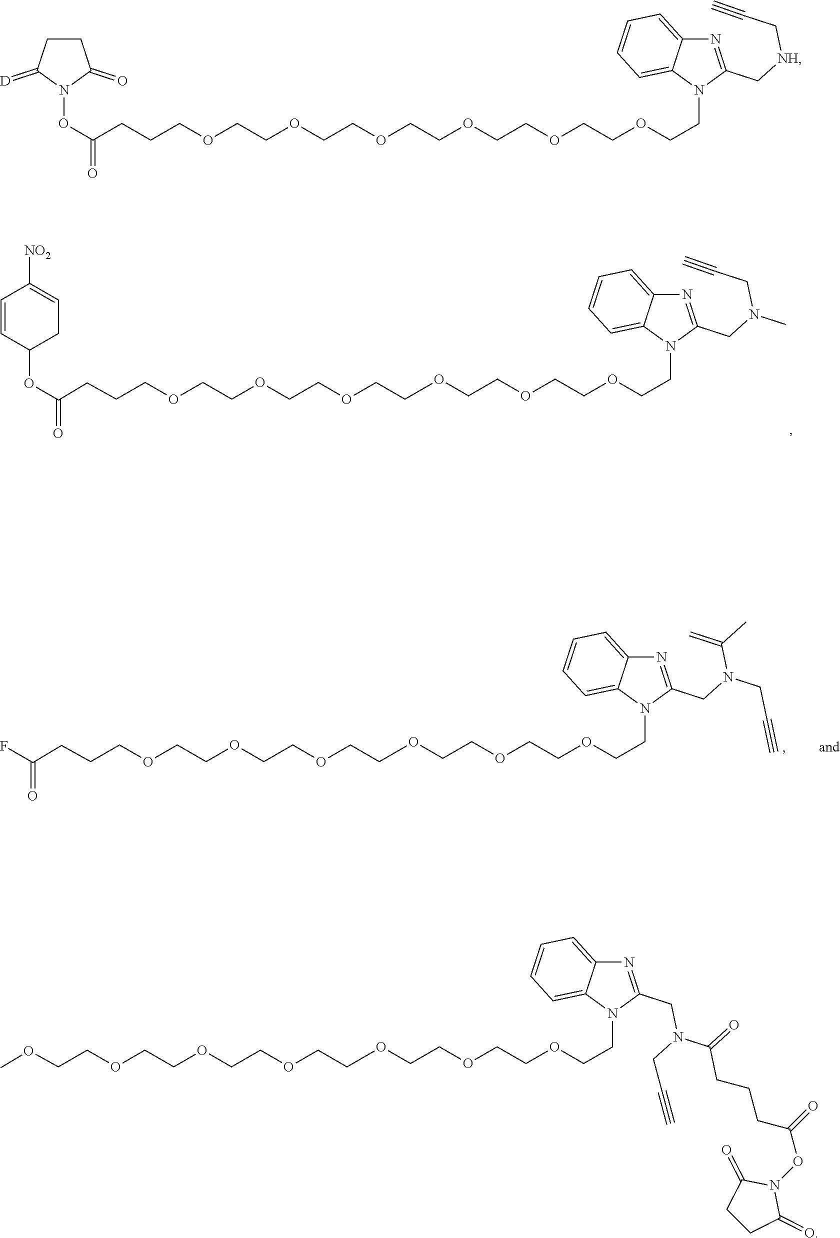

In some embodiments, method of preparing a conjugate of Formula A, comprises
a) contacting an alkyne-containing substance with a compound according to claim 1 under conditions under which said substance and compound shall react with each other;
b) purifying the conjugate or detecting the conjugate in situ by fluorescence.

In some embodiments, the substance is selected from an alkyne-modified antibody, alkyne-modified avidin, alkyne-modified streptavidin, alkyne-modified neutravidin, and alkyne-modified enzyme.

In some embodiments, the substance is an alkyne-labeled cell or tissue.

In some embodiments, a method of preparing a conjugate comprises
a) contacting an azido-containing substance with a compound according to claim 1 under conditions under which said substance and compound shall react;
b) purifying the made conjugate or detecting the made conjugate in situ by fluorescence.

In some embodiments, the substance is selected from an azido-modified antibody, azido-modified avidin, azido-modified streptavidin, azido-modified neutravidin, and azido-modified enzyme.

In some embodiments, the substance is an azido-labeled cell or tissue.

In some embodiments, a method of preparing a conjugate comprises
a) contacting a substance with a compound of Formula A, I, or II under conditions under which said substance and compound shall react;
b) incubating the said modified substance with an alkyne;
c) purifying the made conjugate or detecting the made conjugate in situ by fluorescence.

In some embodiments, a method of preparing a conjugate comprises the steps of:
a) contacting a substance with a compound of Formula A, III, or IV under conditions under which said substrate and compound shall react;
b) incubating the compound of a) with an azido compound;
c) purifying the conjugate or detecting the made conjugate in situ by fluorescence.

In some embodiments, a method of detecting an analyte in a sample comprises the steps of:
a) contacting said sample with a detection reagent comprising a compound or conjugate containing the structure of Formula A, I, II, III or IV under conditions under which said detection reagent will react with the said analyte; and
   b) detecting the detection reagent bound analyte by fluorescence.

The present disclosure disclosed a new type of azido compounds and a new type of alkyne compounds used to for preparing biological conjugates that have (1) high specificity; (2) fast reaction; (3) high water compatibility; and (4) the addition of Cu(I) chelator is not required for initiating the click reactions.

In some embodiments of the disclosure, wherein X is N; Z is O, S, or N—R₈; R₂ is H, carboxy, sulfonate, PEG, alkoxy, aryloxy, aryl or heteroaryl; R₈, R₁₀ and R₁₁ are independently H, an alkyl, an aryl or a heteroaryl; provided that one of R₁ to R₁₁ contains a BDM, a FG, or a L-BS.

In some embodiments of the disclosure, wherein X is CR₁; Z is O, S, or N—R₈; R₁ and R₂, combined to form an aryl or an heteroaryl; R₈, R₁₀ and R₁₁ are independently H, an alkyl, an aryl or a heteroaryl; provided that one of R₁ to R₁₁ contains a BDM, an FG, or a L-BS.

In some embodiments of the disclosure, the compounds contain an FD as a BDG. The fluorescent dyes linked to the compounds of the disclosure are typically an FD. They are typically selected from coumarins, styryls, fluoresceins, rhodamines, cyanines, BODIPYs, or other polycyclic aromatics. Many of them are commercially available as selectively listed in Table 1 as examples.

TABLE 1
The typical fluorescent dyes (FD) that can
be linked to the compounds of Formula 1

| | Absorption | Emission |
|---|---|---|
| Fluorophore | (nm) | (nm) |
| ATTO 465 | 453 | 508 |
| ATTO 488 | 501 | 523 |
| ATTO 495 | 495 | 527 |
| ATTO 514 | 511 | 533 |
| ATTO 532 | 532 | 553 |
| ATTO 550 | 554 | 576 |
| ATTO 565 | 563 | 592 |
| ATTO 590 | 594 | 624 |
| ATTO 594 | 601 | 627 |
| ATTO 610 | 615 | 634 |
| ATTO 620 | 619 | 643 |
| ATTO 633 | 629 | 657 |
| ATTO 647 | 645 | 669 |
| ATTO 647N | 644 | 669 |
| ATTO 655 | 663 | 684 |
| ATTO 665 | 663 | 684 |
| ATTO 680 | 680 | 700 |
| ATTO 700 | 700 | 719 |
| ATTO 725 | 729 | 752 |
| ATTO 740 | 740 | 764 |
| 5-carboxy-2,7-dichlorofluorescein | 504 | 529 |
| 5-Carboxyfluorescein (5-FAM) | 492 | 518 |
| 5-Carboxynapthofluorescein | 598 | 668 |
| 5-Carboxytetramethylrhodamine | 542 | 568 |
| (5-TAMRA) | | |
| 5-FAM (5-Carboxyfluorescein) | 492 | 518 |
| 5-ROX | 578 | 604 |
| 6-TAMRA | 548 | 568 |
| 6-Carboxyrhodamine 6G | 518 | 543 |
| 6-CR6G | 518 | 543 |
| 6-JOE | 520 | 548 |
| 6-FAM | 494 | 517 |
| 6-ROX | 570 | 591 |
| Alexa Fluor 488 | 492 | 520 |
| Alexa Fluor 532 | 532 | 554 |
| Alexa Fluor 546 | 557 | 573 |
| Alexa Fluor 568 | 578 | 603 |
| Alexa Fluor 594 | 594 | 618 |
| Alexa Fluor 633 | 632 | 650 |
| Alexa Fluor 647 | 647 | 666 |
| Alexa Fluor 660 | 668 | 698 |
| Alexa Fluor 680 | 679 | 702 |
| Bodipy 492/515 | 490 | 515 |
| Bodipy 493/503 | 533 | 549 |
| Bodipy 500/510 | 509 | 515 |
| Bodipy 505/515 | 502 | 510 |
| Bodipy 530/550 | 528 | 547 |
| Bodipy 542/563 | 543 | 563 |
| Bodipy 558/568 | 558 | 569 |
| Bodipy 564/570 | 564 | 570 |
| Bodipy 576/589 | 579 | 590 |
| Bodipy 581/591 | 584 | 592 |
| Bodipy 630/650-X | 625 | 642 |
| Bodipy 650/665-X | 647 | 665 |
| Bodipy 665/676 | 605 | 676 |
| Bodipy Fl | 505 | 513 |
| Bodipy R6G SE | 528 | 547 |
| Bodipy TMR | 542 | 574 |
| Bodipy TR | 589 | 617 |
| CF 488A | 490 | 515 |
| CF 555 | 555 | 565 |
| CF 568 | 562 | 583 |
| CF 594 | 593 | 614 |
| CF 633 | 630 | 650 |
| CF 640R | 642 | 662 |
| CF 647 | 650 | 665 |
| CF 660C | 667 | 685 |
| CF 680 | 681 | 698 |
| CF680R | 680 | 701 |
| CF 750 | 755 | 777 |
| CF 770 | 770 | 797 |
| CF 790 | 784 | 806 |
| CL-NERF | 504 | 540 |
| CMFDA | 494 | 520 |
| Cy2 | 489 | 506 |
| Cy3 | 554 | 568 |
| Cy3.5 | 581 | 598 |
| Cy5 | 649 | 666 |
| Cy5.5 | 675 | 695 |
| Cy7 | 743 | 767 |
| DDAO | 646 | 659 |
| DiA | 456 | 591 |
| DiD | 644 | 665 |
| DiI | 549 | 565 |
| DyLight 488 | 493 | 518 |
| DyLight 550 | 562 | 576 |
| DyLight 594 | 593 | 618 |
| DyLight 633 | 638 | 658 |
| DyLight 650 | 652 | 672 |
| DyLight 680 | 692 | 712 |
| DyLight 755 | 754 | 776 |
| DyLight 800 | 777 | 794 |
| DiO | 487 | 502 |
| DiR | 748 | 780 |
| DM-NERF | 497 | 540 |
| DsRed | 558 | 583 |
| DTAF | 494 | 520 |
| DY-490 | 491 | 515 |
| DY-495 | 494 | 521 |
| DY-505 | 507 | 528 |
| DY-530 | 533 | 554 |
| DY-547 | 558 | 573 |
| DY-548 | 558 | 572 |
| DY-549 | 562 | 577 |
| DY-549P1 | 563 | 578 |
| DY-550 | 562 | 577 |
| DY-554 | 544 | 570 |
| DY-555 | 547 | 573 |
| DY-556 | 548 | 574 |
| DY-560 | 560 | 578 |
| DY-590 | 581 | 600 |
| DY-591 | 581 | 598 |
| DY-594 | 594 | 615 |
| DY-605 | 600 | 624 |
| DY-610 | 610 | 632 |
| DY-615 | 623 | 643 |
| DY-630 | 638 | 658 |
| DY-631 | 637 | 657 |
| DY-632 | 636 | 658 |
| DY-633 | 638 | 658 |
| DY-634 | 636 | 657 |
| DY-635 | 648 | 670 |
| DY-636 | 647 | 670 |
| DY-647 | 653 | 673 |
| DY-648 | 655 | 676 |
| DY-649 | 656 | 670 |
| DY-649P1 | 654 | 672 |
| DY-650 | 656 | 676 |
| DY-651 | 655 | 677 |
| DY-652 | 653 | 676 |
| DY-654 | 653 | 677 |
| DY-675 | 675 | 699 |
| DY-676 | 675 | 699 |
| DY-677 | 674 | 698 |
| DY-678 | 674 | 694 |
| DY-679 | 679 | 698 |
| DY-679P1 | 679 | 697 |
| DY-680 | 691 | 709 |
| DY-681 | 692 | 709 |
| DY-682 | 692 | 709 |
| DY-700 | 707 | 728 |
| DY-701 | 709 | 730 |
| DY-703 | 705 | 721 |
| DY-704 | 706 | 721 |
| DY-730 | 734 | 755 |
| DY-731 | 736 | 755 |
| DY-732 | 735 | 756 |
| DY-734 | 733 | 755 |
| DY-749 | 759 | 780 |
| DY-750 | 751 | 774 |
| DY-751 | 752 | 772 |
| DY-752 | 750 | 771 |
| DY-754 | 748 | 771 |
| DY-776 | 772 | 787 |
| DY-777 | 770 | 788 |
| DY-778 | 767 | 787 |
| DY-780 | 783 | 799 |
| DY-781 | 784 | 796 |
| DY-782 | 785 | 794 |
| DY-800 | 777 | 791 |
| DY-831 | 844 | 875 |
| Eosin | 524 | 545 |
| Erythrosin | 529 | 555 |
| FITC | 490 | 520 |
| Fluo-3 | 506 | 520 |
| Fluo-4 | 494 | 516 |
| Fluor-Ruby | 555 | 582 |
| FluorX | 494 | 520 |
| FM 1-43 | 479 | 598 |
| FM 4-46 | 515 | 640 |
| iFluor 488 | 498 | 520 |
| iFluor 555 | 558 | 578 |
| iFluor 594 | 588 | 610 |
| iFluor 647 | 649 | 670 |
| iFluor 680 | 686 | 702 |
| iFluor 700 | 696 | 720 |
| iFluor 750 | 755 | 785 |
| iFluor 780 | 787 | 808 |
| Lyso Tracker Green | 504 | 511 |
| Lyso Tracker Yellow | 551 | 576 |
| Mitotracker Green | 490 | 516 |
| Mitotracker Orange | 551 | 576 |
| Mitotracker Red | 578 | 599 |
| NBD | 466 | 539 |
| Oregon Green 488 | 494 | 517 |
| Oregon Green 514 | 506 | 526 |
| PKH26 | 551 | 567 |
| PKH67 | 496 | 520 |
| Resorufin | 571 | 584 |
| RH 414 | 532 | 716 |
| Rhod-2 | 552 | 576 |
| Rhodamine | 550 | 573 |
| Rhodamine 110 | 496 | 520 |
| Rhodamine 123 | 507 | 529 |
| Rhodamine 6G | 525 | 555 |
| Rhodamine B | 540 | 625 |
| Rhodamine Green | 502 | 527 |
| Rhodamine Red | 570 | 590 |
| Rose Bengal | 525 | 550 |
| Spectrum Green | 497 | 538 |
| Spectrum Orange | 559 | 588 |
| Spectrum Red | 587 | 512 |
| SYTO 11 | 508 | 527 |
| SYTO 12 | 499 | 522 |
| SYTO 13 | 488 | 509 |
| SYTO 14 | 517 | 549 |
| SYTO 15 | 516 | 546 |
| SYTO 16 | 488 | 518 |
| SYTO 17 | 621 | 634 |
| SYTO 18 | 490 | 507 |
| SYTO 20 | 512 | 530 |
| SYTO 21 | 494 | 517 |
| SYTO 22 | 515 | 535 |
| SYTO 23 | 499 | 520 |
| SYTO 24 | 490 | 515 |
| SYTO 25 | 521 | 556 |
| SYTO 40 | 420 | 441 |
| SYTO 41 | 430 | 454 |
| SYTO 42 | 433 | 460 |
| SYTO 43 | 436 | 467 |
| SYTO 44 | 446 | 471 |
| SYTO 45 | 452 | 484 |
| SYTO 59 | 622 | 645 |
| SYTO 60 | 652 | 678 |
| SYTO 61 | 628 | 645 |
| SYTO 62 | 652 | 676 |
| SYTO 63 | 657 | 673 |
| SYTO 64 | 599 | 619 |
| SYTO 80 | 531 | 545 |
| SYTO 81 | 530 | 544 |
| SYTO 82 | 541 | 560 |
| SYTO 83 | 543 | 559 |
| SYTO 84 | 567 | 582 |
| SYTO 85 | 567 | 583 |
| SYTOX Blue | 445 | 470 |
| SYTOX Green | 504 | 523 |
| SYTOX Orange | 547 | 570 |
| Texas Red | 595 | 620 |
| Tide Fluor 2 (TF2) | 500 | 527 |
| Tide Fluor 2WS (TF2WS) | 502 | 525 |
| Tide Fluor 3 (TF3) | 555 | 584 |
| Tide Fluor 3WS(TF3WS) | 555 | 565 |
| Tide Fluor 4 (TF4) | 590 | 618 |
| Tide Fluor 5WS (TF5WS) | 649 | 664 |
| Tide Fluor 6WS (TF6WS) | 676 | 695 |
| Tide Fluor 7WS (TF7WS) | 749 | 775 |
| Tide Fluor 8WS (TF8WS) | 775 | 807 |
| TRITC | 550 | 573 |
| XTRITC | 582 | 601 |

Many embodiments of the compounds of the disclosure possess an overall electronic charge. It is to be understood that when such electronic charges are shown to be present, they are balanced by the presence of appropriate counterions, which may or may not be explicitly identified. A biologically compatible counterion, which is preferred for some applications, is not toxic in biological applications, and does not have a substantially deleterious effect on biomolecules. Where the compound of the disclosure is positively charged, the counterion is typically selected from, but not limited to, chloride, bromide, iodide, sulfate, alkanesulfonate, arylsulfonate, phosphate, perchlorate, tetrafluoroborate, tetraarylboride, nitrate and anions of aromatic or aliphatic carboxylic acids. Where the compound of the disclosure is negatively charged, the counterion is typically selected from, but not limited to, alkali metal ions, alkaline earth metal ions, transition metal ions, ammonium or substituted ammonium or pyridinium ions. Preferably, any necessary counterion is biologically compatible, is not toxic as used, and does not have a substantially deleterious effect on biomolecules. Counterions are readily changed by methods well known in the art, such as ion-exchange chromatography, or selective precipitation.

It is to be understood that the compounds of the disclosure have been drawn in one or another particular electronic resonance structure. Every aspect of the instant disclosure applies equally to compounds that are formally drawn with other permitted resonance structures, as the electronic charge on the subject compounds is delocalized throughout the compound conjugate itself.

In some embodiments of the disclosure, the compounds contain a FG. The typical functional groups linked to the compounds of the disclosure are listed in Table 2 as examples.

In some embodiments of the disclosure, the compound conjugate contains at least one L-BS, where BS attached to the compound by a well-known reaction as listed in Table 2 as examples. In certain embodiments, the covalent linkage attaching the compound to BS contains multiple intervening atoms that serve as a Linker (L). The compounds can be used to label a wide variety of biological, organic or inorganic substances that contain or are modified to contain functional groups with suitable reactivity, resulting in chemical attachment of the conjugated substances.

TABLE 2
Examples of functional groups for
preparing the biological conjugates

| Functional | Matching | Resulting |
|---|---|---|
| Group (FG) | Functional Group | covalent linkages |
| Amines | Carbonyl | Carboxamides/Shift bases |
| activated esters | amines/anilines | carboxamides |
| acrylamides | thiols | thioethers |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thioethers |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |
| hydroxylamines | aldehydes/ketones | oxamines |
| hydrazines | aldehydes/ketones | hydrazones |
| cysteines | aldehydes/ketones | thiazolidines |
| Nitrile-N-oxides | cycloalkynes | isoxazoles |
| anthracenes | maleimides | succinimides |

Choice of the linkage used to attach the compound to a biological substrate to be conjugated typically depends on the functional group on the biological substrate to be conjugated and the type or length of covalent linkage desired. The types of functional groups typically present on the organic or inorganic biological substrates include, but are not limited to, amines, amides, thiols, alcohols, phenols, aldehydes, ketones, phosphonates, imidazoles, hydrazines, hydroxylamines, disubstituted amines, halides, epoxides, carboxylate esters, sulfonate esters, purines, pyrimidines, carboxylic acids, olefinic bonds or a combination of these groups. A single type of reactive site may be available on the biological substrate (typical for polysaccharides), or a variety of sites may occur (e.g. amines, thiols, alcohols, phenols), as is typical for proteins. A conjugated biological substrate may be conjugated to more than one compound conjugate, which may be the same or different, or to a biological substrate that is additionally modified by a hapten, such as biotin. Alternatively multiple substrates might be conjugated to a single compound. Although some selectivity can be obtained by careful control of the reaction conditions, selectivity of labeling is best obtained by selection of an appropriate reactive compound conjugate.

Typically, compound will react with an amine, a thiol, an alcohol, an aldehyde or a ketone. Preferably compound reacts with an amine or a thiol. In one embodiment, compound is an acrylamide, a reactive amine (including a cadaverine or ethylenediamine), an activated ester of a carboxylic acid (typically a succinimidyl ester of a carboxylic acid), an acyl nitrile, an aldehyde, an alkyl halide, an anhydride, an aniline, an aryl halide, an aziridine, a boronate, a carboxylic acid, a diazoalkane, a haloacetamide, a halotriazine, a hydrazine (including hydrazides), an imido ester, an isocyanate, an isothiocyanate, a maleimide, a phosphoramidite, a reactive platinum complex, a sulfonyl halide, or a thiol group. By “reactive platinum complex” is particularly meant chemically reactive platinum complexes such as described in U.S. Pat. Nos. 5,580,990; 5,714,327 and 5,985,566.

Where the compound is a photoactivatable, such as diazirinyl or psoralen derivative, the compound becomes chemically reactive only after illumination with light of an appropriate wavelength. Where compound is an activated ester of a carboxylic acid, the reactive compound is particularly useful for preparing compounds of proteins, nucleotides, oligonucleotides, or haptens. Where compound is a maleimide or haloacetamide the reactive compound is particularly useful for conjugation to thiol-containing biological substrates. Where compound is a hydrazide, the reactive compound is particularly useful for conjugation to periodate-oxidized carbohydrates and glycoproteins, and in addition is an aldehyde-fixable polar tracer for cell microinjection. Preferably, compounds are a carboxylic acid, a succinimidyl ester of a carboxylic acid, a haloacetamide, a hydrazine, an isothiocyanate, a maleimide group, an aliphatic amine, a perfluorobenzamido, or a psoralen. Preferably, compound is a succinimidyl ester of a carboxylic acid, a maleimide, an iodoacetamide, or a reactive platinum complex.

Based on the above-mentioned attributes, the appropriate reactive compounds of the disclosure are selected for the preparation of the desired compounds, whose advantageous properties make them useful for a wide variety of applications. Particularly useful compounds include, among others, conjugates where substrate is a peptide, a nucleotide, an antigen, a steroid, a vitamin, a drug, a hapten, a metabolite, a toxin, an environmental pollutant, an amino acid, a protein, a nucleic acid, a nucleic acid compound, a carbohydrate, a lipid, an ion-complexing moiety, a glass or a non-biological compound. Alternatively, substrate is a cell, a cellular system, a cellular fragment, or a subcellular particle (e.g. inter alia), a virus particle, a bacterial particle, a virus component, a biological cell (such as animal cell, plant cell, bacteria, yeast, or protist), or a cellular component. Reactive compounds typically label functional groups at the cell surface, in cell membranes, organelles, or cytoplasm.

Typically substrate is an amino acid, a peptide, a protein, a tyramine, a polysaccharide, an ion-complexing moiety, a nucleoside, a nucleotide, an oligonucleotide, a nucleic acid, a hapten, a psoralen, a drug, a hormone, a lipid, a lipid assembly, a compound, a compound microparticle, a biological cell or virus. More typically, substrate is a peptide, a protein, a nucleotide, an oligonucleotide, or a nucleic acid. When conjugating compounds of the disclosure to such compounds, it is possible to incorporate more compounds per molecule to increase the fluorescent signal. For compound-antibody conjugates, one compound/antibody is preferred.

In one embodiment, substrate is an amino acid (including those that are protected or are substituted by phosphonates, carbohydrates, or C₁ to C₂₅ carboxylic acids), or is a compound of amino acids such as a peptide or protein. Preferred conjugates of peptides contain at least five amino acids, preferably 5 to 36 amino acids. Preferred peptides include, but are not limited to, neuropeptides, cytokines, toxins, protease substrates, and protein kinase substrates. Preferred protein conjugates include enzymes, antibodies, lectins, glycoproteins, histones, albumins, lipoproteins, avidin, streptavidin, protein A, protein G, phycobiliproteins and other fluorescent proteins, hormones, toxins, chemokines and growth factors. In one preferred aspect, the conjugated protein is a compound antibody conjugate.

In one aspect of the disclosure, substrate is a conjugated biological substrate that is an antibody (including intact antibodies, antibody fragments, and antibody sera, etc.), an amino acid, an angiostatin or endostatin, an avidin or streptavidin, a biotin (e.g. an amidobiotin, a biocytin, a desthiobiotin, etc.), a blood component protein (e.g. an albumin, a fibrinogen, a plasminogen, etc.), a dextran, an enzyme, an enzyme inhibitor, an IgG-binding protein (e.g. a protein A, protein G, protein A/G, etc.), a fluorescent protein (e.g. a phycobiliprotein, an aequorin, a green fluorescent protein, etc.), a growth factor, a hormone, a lectin (e.g. a wheat germ agglutinin, a conconavalin A, etc.), a lipopolysaccharide, a metal-binding protein (e.g. a calmodulin, etc.), a microorganism or portion thereof (e.g. a bacteria, a virus, a yeast, etc.), a neuropeptide and other biologically active factors (e.g. a dermorphin, a deltropin, an endomorphin, an endorphin, a tumor necrosis factor etc.), a non-biological microparticle (e.g. of ferrofluid, gold, polystyrene, etc.), a nucleotide, an oligonucleotide, a peptide toxin (e.g. an apamin, a bungarotoxin, a phalloidin, etc.), a phospholipid-binding protein (e.g. an annexin, etc.), a small-molecule drug (e.g. a methotrexate, etc.), a structural protein (e.g. an actin, a fibronectin, a laminin, a microtubule-associated protein, a tublin, etc.), or a tyramide.

In some embodiments, substrate is a nucleic acid base, nucleoside, nucleotide or a nucleic acid compound, including those that are modified to possess an additional linker or spacer for attachment of the compounds of the disclosure, such as an alkynyl linkage (U.S. Pat. No. 5,047,519), an aminoallyl linkage (U.S. Pat. No. 4,711,955), or a heteroatom-substituted linker (U.S. Pat. No. 5,684,142) or other linkage. In some embodiments, the conjugated biological substrate is a nucleoside or nucleotide analog that links a purine or pyrimidine base to a phosphate or polyphosphate moiety through a noncyclic spacer. In some embodiments, the compound conjugate is conjugated to the carbohydrate portion of a nucleotide or nucleoside, typically through a hydroxyl group but additionally through a thiol or an amino group (U.S. Pat. Nos. 5,659,025; 5,668,268; 5,679,785). Typically, the conjugated nucleotide is a nucleoside triphosphate or a deoxynucleoside triphosphate or a dideoxynucleoside triphosphate. Incorporation of methylene moieties or nitrogen or sulfur heteroatoms into the phosphate or polyphosphate moiety is also useful. Nonpurine and nonpyrimidine bases such as 7-deazapurines (U.S. Pat. No. 6,150,510) and nucleic acids containing such bases can also be coupled to compounds of the disclosure. Nucleic acid adducts prepared by reaction of depurinated nucleic acids with amine, hydrazide or hydroxylamine derivatives provide an additional means of labeling and detecting nucleic acids, e.g. “A method for detecting abasic sites in living cells: age-dependent changes in base excision repair.” Atamna H, Cheung I, Ames B N. PROC. NATL. ACAD. SCI. U.S.A. 97, 686-691 (2000).

Preferred nucleic acid compounds are labeled, single- or multi-stranded, natural or synthetic DNA or RNA, DNA or RNA oligonucleotides, or DNA/RNA hybrids, or incorporate an unusual linker such as morpholine derivatized phosphates, or peptide nucleic acids such as N-(2-aminoethyl)glycine units. When the nucleic acid is a synthetic oligonucleotide, it typically contains fewer than 50 nucleotides, more typically fewer than 25 nucleotides. Conjugates of peptide nucleic acids (PNA) (Nielsen, et al. U.S. Pat. No. 5,539,082) may be preferred for some applications because of their generally faster hybridization rates.

In one embodiment, the conjugated oligonucleotides of the disclosure are aptamers for a particular target molecule, such as a metabolite, compound conjugate, hapten, or protein. That is, the oligonucleotides have been selected to bind preferentially to the target molecule. Methods of preparing and screening aptamers for a given target molecule have been previously described and are known in the art [for example, U.S. Pat. No. 5,567,588 to Gold (1996)].

In some embodiments, substrate is a carbohydrate that is typically a polysaccharide, such as a dextran, heparin, glycogen, amylopectin, mannan, inulin, starch, agarose and cellulose. Alternatively, the carbohydrate is a polysaccharide that is a lipopolysaccharide. Preferred polysaccharide conjugates are dextran, or lipopolysaccharide conjugates.

Conjugates having an ion-complexing moiety serve as indicators for calcium, sodium, magnesium, zinc, potassium, or other biologically important metal ions. Preferred ion-complexing moieties are crown ethers (U.S. Pat. No. 5,405,975); derivatives of 1,2-bis-(2-aminophenoxyethane)-N,N,N′,N′-tetraacetic acid (BAPTA chelators; U.S. Pat. Nos. 5,453,517; 5,516,911 and 5,049,673); derivatives of 2-carboxymethoxyaniline-N,N-di-acetic acid (APTRA chelators; AM. J. PHYSIOL., 256, C540 (1989)); or pyridine- and phenanthroline-based metal ion chelators (U.S. Pat. No. 5,648,270): or derivatives of nitrilotriacetic acid, see e.g. “Single-step synthesis and characterization of biotinylated nitrilotriacetic acid, a unique reagent for the detection of histidine-tagged proteins immobilized on nitrocellulose”, McMahan S A and Burgess R R, ANAL. BIOCHEM., 236, 101-106 (1996). Preferably, the ion-complexing moietZ is a crown ether chelator, a BAPTA chelator, an APTRA chelator or a derivative of nitrilotriacetic acid.

Other conjugates of non-biological materials include compound conjugate-conjugates of organic or inorganic compounds, compound films, compound wafers, compound membranes, compound particles, or compound microparticles (magnetic and non-magnetic microspheres); iron, gold or silver particles; conducting and non-conducting metals and non-metals; and glass and plastic surfaces and particles. In some embodiments, the conjugated biological substrate is a glass or silica, which may be formed into an optical fiber or other structure.

In one embodiment, conjugates of biological compounds such as peptides, proteins, oligonucleotides, nucleic acid compounds are also labeled with at least a second fluorescent dye conjugate, which is optionally an additional compound conjugate of the present disclosure, to form an energy-transfer pair. In some aspects of the disclosure, the labeled conjugate functions as an enzyme substrate, and enzymatic hydrolysis disrupts the energy transfer. In some embodiments of the disclosure, the energy-transfer pair that incorporates a compound conjugate of the disclosure is conjugated to an oligonucleotide that displays efficient fluorescence quenching in its hairpin conformation [the so-called “molecular beacons” of Tyagi, et al., NATURE BIOTECHNOLOGY, 16, 49 (1998)] or fluorescence energy transfer.

The preparation of compounds using reactive compounds is well documented, e.g. U.S. Pat. Nos. 8,158,444; 8,455,613; 8,354,239; 8,362,193; and U.S. Pat. No. 8,575,303 to Gaylord, et al., also WO 2013/101902 to Chiu et al. The other biological applications of polyconjugated compounds have been well documented by Thomas Ill et al. (Chem. Rev. 2007, 107, 1339); Zhu et al (Chem. Rev. 2012, 112, 4687) and Zhu et al. (Chem. Soc. Rev., 2011, 40, 3509). Conjugates typically result from mixing appropriate reactive compounds and biological substrate to be conjugated in a suitable solvent in which both are soluble. The compounds of the disclosure are readily soluble in aqueous solutions, facilitating conjugation reactions with most biological materials. For those reactive compounds that are photoactivated, conjugation requires illumination of the reaction mixture to activate the reactive compounds.

In some embodiments, the disclosure provides the compounds of Formula IV, wherein BS is an antibody, an antigen, a protein, a peptide, an oligonucleotide, a DNA, an RNA, a PNA, an aptamer or a cell.

The present disclosure provides a method of preparing a conjugate, comprising
a) mixing an alkyne-containing substance with a compound of Formula A, under conditions under which said substance and compound shall react,
b) incubating the reaction mixture to maximize the conjugation yield;
c) Purifying the made conjugate or detecting the made conjugate in situ by fluorescence.

In some embodiments, wherein the substance is an alkyne-modified antibody, an alkyne-modified avidin, an alkyne-modified streptavidin, an alkyne-modified neutravidin, or an alkyne-modified enzyme.

In some embodiments, wherein the substance is an alkyne-labeled cell or tissue.

The present disclosure provides a method of preparing a conjugate, comprising
a) Modifying a substance with a compound of Formula A under conditions under which said substance and compound shall react;
b) incubating the said modified substance with an alkyne;
c) Purifying the made conjugate or detecting the made conjugate in situ by fluorescence.

In some embodiments, wherein the substance is an antibody, an avidin, or an enzyme.

In some embodiments, wherein the substance is a cell or tissue.

The present disclosure provides a method of preparing a conjugate, comprising
a) mixing an azido-containing substance with a compound of Formula A under conditions under which said substance and compound shall react;
b) incubating the reaction mixture to maximize the conjugation yield;
c) Purifying the made conjugate or detecting the made conjugate in situ by fluorescence.

In some embodiments, wherein the substance is an azido-modified antibody, an azido-modified avidin, an azido-modified streptavidin, an azido-modified neutravidin, or an azido-modified enzyme.

In some embodiments, wherein the substance is an azido-labeled cell or tissue.

The present disclosure provides a method of preparing a conjugate, comprising
a) Modifying a biological substrate with a compound of Formula A under conditions under which said substrate and compound shall react;
b) incubating the said modified substrate with an azido compound;
c) Purifying the made conjugate or detecting the made conjugate in situ by fluorescence.

In some embodiments, wherein the substance is an antibody, an avidin, or an enzyme.

In some embodiments, wherein the substance is a cell or tissue.

The present disclosure further provides a method of detecting an analyte in a sample, comprising
a) combining said sample with a detection reagent comprising a compound conjugate containing the structure of Formula A, 1, 2, 3 or 4 under conditions under which said detection reagent will react with the said analyte; and
b) detecting the detection reagent bound analyte by fluorescence.

In one embodiment, the disclosure provides the compound conjugate of Formula A, 1, 2, 3 or 4 wherein BS is an antibody.

In some embodiments, the disclosure provides the compound conjugate of Formula A, 1, 2, 3 or 4 wherein BS is an anti-digoxigenin antibody.

In some embodiments, the disclosure provides the compound conjugate of Formula A, 1, 2, 3 or 4 wherein BS is a goat anti-mouse IgG antibody, goat anti-rabbit IgG antibody, goat anti-human IgG antibody, donkey anti-mouse IgG antibody, donkey anti-rabbit IgG antibody, donkey anti-human IgG antibody, chicken anti-mouse IgG antibody, chicken anti-rabbit IgG antibody, or chicken anti-human IgG antibody.

In some embodiments, the disclosure provides the compound conjugate of Formula A, 1, 2, 3 or 4 wherein BS is an avidin, streptavidin, neutravidin, avidin, or avidin.

In some embodiments, the disclosure provides the compound conjugate of Formula A, 1, 2, 3 or 4 wherein the analyte is a target protein expressed on a cell surface.

In some embodiments, the sample is present on or in solid or semi-solid matrix. In one aspect of the disclosure, the matrix is a membrane.

In another aspect, the matrix is an electrophoretic gel, such as is used for separating and characterizing nucleic acids or proteins, or is a blot prepared by transfer from an electrophoretic gel to a membrane.

In another aspect, the matrix is a silicon chip or glass slide, and the analyte of interest has been immobilized on the chip or slide in an array (e.g. the sample comprises proteins or nucleic acid compounds in a microarray). In yet another aspect, the matrix is a microwell plate or microfluidic chip, and the sample is analyzed by automated methods, typically by various methods of high-throughput screening, such as drug screening.

The compounds of the disclosure are generally utilized by combining a compound conjugate of the disclosure as described above with the sample of interest under conditions selected to yield a detectable optical response. The term “compound conjugate” is used herein to refer to all aspects of the claimed compounds. The compound conjugate typically forms a covalent association or complex with an element of the sample, or is simply present within the bounds of the sample or portion of the sample. The sample is then illuminated at a wavelength selected to elicit the optical response. Typically, staining the sample is used to determine a specified characteristic of the sample by further comparing the optical response with a standard or expected response.

A detectable optical response means a change in, or occurrence of, an optical signal that is detectable either by observation or instrumentally. Typically, the detectable response is a change in fluorescence, such as a change in the intensity, excitation or emission wavelength distribution of fluorescence, fluorescence lifetime, fluorescence polarization, or a combination thereof. The degree and/or location of staining, compared with a standard or expected response, indicates whether and to what degree the sample possesses a given characteristic.

For biological applications, the compounds of the disclosure are typically used in an aqueous, mostly aqueous or aqueous-miscible solution prepared according to methods generally known in the art. The exact concentration of compound conjugate is dependent upon the experimental conditions and the desired results. The optimal concentration is determined by systematic variation until satisfactory results with minimal background fluorescence are accomplished.

The compounds are most advantageously used to stain samples with biological components. The sample may comprise heterogeneous mixtures of components (including intact cells, cell extracts, bacteria, viruses, organelles, and mixtures thereof), or a single component or homogeneous group of components (e.g. natural or synthetic amino acids, nucleic acids or carbohydrate compounds, or lipid membrane complexes). These compounds are generally non-toxic to living cells and other biological components, within the concentrations of use.

The compound conjugate is combined with the sample in any way that facilitates contact between the compound conjugate and the sample components of interest. Typically, the compound conjugate or a solution containing the compound conjugate is simply added to the sample. Certain compounds of the disclosure tend to be impermeant to membranes of biological cells, and once inside viable cells are typically well retained. Treatments that permeabilize the plasma membrane, such as electroporation, shock treatments or high extracellular ATP can be used to introduce selected compounds into cells. Alternatively, selected compounds can be physically inserted into cells, e.g. by pressure microinjection, scrape loading, patch clamp methods, or phagocytosis.

Compounds that incorporate an aliphatic amine or a hydrazine residue can be microinjected into cells, where they can be fixed in place by aldehyde fixatives such as formaldehyde or glutaraldehyde. This fixability makes such compounds useful for intracellular applications such as neuronal tracing.

Compounds that possess a lipophilic substituent, such as phospholipids, will non-covalently incorporate into lipid assemblies, e.g. for use as probes for membrane structure; or for incorporation in liposomes, lipoproteins, films, plastics, lipophilic microspheres or similar materials; or for tracing. Lipophilic compounds are useful as fluorescent probes of membrane structure.

Using compounds to label active sites on the surface of cells, in cell membranes or in intracellular compartments such as organelles, or in the cell's cytoplasm, permits the determination of their presence or quantity, accessibility, or their spatial and temporal distribution in the sample. Photoreactive compounds can be used similarly to photolabel components of the outer membrane of biological cells or as photo-fixable polar tracers for cells.

Optionally, the sample is washed after staining to remove residual, excess or unbound compound conjugate. The sample is optionally combined with one or more other solutions in the course of staining, including wash solutions, permeabilization and/or fixation solutions, and solutions containing additional detection reagents. An additional detection reagent typically produces a detectable response due to the presence of a specific cell component, intracellular biological substrate, or cellular condition, according to methods generally known in the art. Where the additional detection reagent has, or yields a product with, spectral properties that differ from those of the subject compounds, multi-color applications are possible. This is particularly useful where the additional detection reagent is a compound conjugate or compound conjugate-conjugate of the present disclosure having spectral properties that are detectably distinct from those of the staining compound conjugate.

The compounds of the disclosure are used according to methods extensively known in the art; e.g. use of antibody conjugates in microscopy and immunofluorescent assays; and nucleotide or oligonucleotide conjugates for nucleic acid hybridization assays and nucleic acid sequencing (e.g., U.S. Pat. No. 5,332,666 to Prober, et al. (1994); U.S. Pat. No. 5,171,534 to Smith, et al. (1992); U.S. Pat. No. 4,997,928 to Hobbs (1991); and WO Appl. 94/05688 to Menchen, et al.). Compound conjugate-conjugates of multiple independent compounds of the disclosure possess utility for multi-color applications.

At any time after or during staining, the sample is illuminated with a wavelength of light selected to give a detectable optical response and observed with a means for detecting the optical response. Equipment that is useful for illuminating the compounds of the disclosure includes, but is not limited to, hand-held ultraviolet lamps, mercury arc lamps, xenon lamps, lasers and laser diodes. These illumination sources are optionally integrated into laser scanners, fluorescence microplate readers, standard or minifluorometers, or chromatographic detectors. Preferred embodiments of the disclosure are compounds that are excitable at or near the wavelengths 405 nm.

The optical response is optionally detected by visual inspection, or by use of any of the following devices: CCD cameras, video cameras, photographic films, laser-scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, examination of the sample optionally includes sorting portions of the sample according to their fluorescence response.

One aspect of the instant disclosure is the formulation of kits that facilitate the practice of various assays using any of the compounds of the disclosure, as described above. The kits of the disclosure typically comprise a fluorescent compound conjugate of the disclosure where the conjugated biological substrate is a specific binding pair member, or a nucleoside, a nucleotide, an oligonucleotide, a nucleic acid compound, a peptide, or a protein. The kit optionally further comprises one or more buffering agents, typically present as an aqueous solution. The kits of the disclosure optionally further comprise additional detection reagents, a purification medium for purifying the resulting labeled biological substrate, luminescence standards, enzymes, enzyme inhibitors, organic solvent, or instructions for carrying out an assay of the disclosure.

### SOME EMBODIMENTS OF THE PRESENT DISCLOSURE

Selected examples of the compounds of the disclosure are provided in Table 3.

TABLE 3
Selected embodiment of the compounds of the disclosure

| Compound # | Chemical Structure |
|---|---|
| 20 | 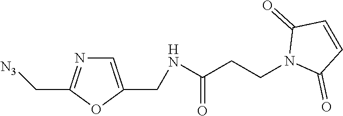 [SMILES: [N-]=[N+]=NCc1ncc(CNC(=O)CCN2C(=O)C=CC2=O)o1] |
| 21 | 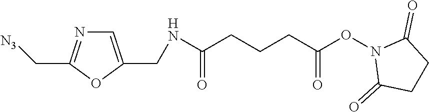 [SMILES: [N-]=[N+]=NCc1ncc(CNC(=O)CCCC(=O)ON2C(=O)CCC2=O)o1] |
| 22 | 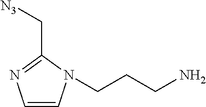 [SMILES: [N-]=[N+]=NCc1nccn1CCCN] |
| 23 | 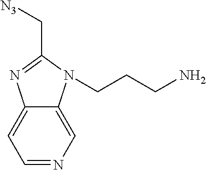 [SMILES: [N-]=[N+]=NCc1nc2ccncc2n1CCCN] |
| 24 | 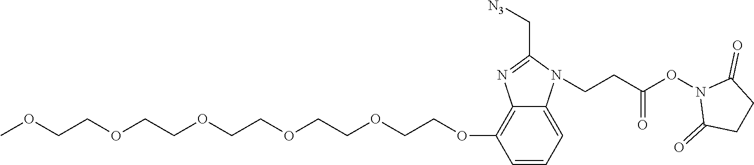 [SMILES: COCCOCCOCCOCCOCCOc1cccc2c1nc(CN=[N+]=[N-])n2CCC(=O)ON1C(=O)CCC1=O] |
| 25 | 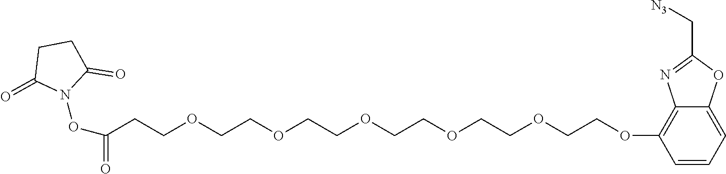 [SMILES: [N-]=[N+]=NCc1nc2c(OCCOCCOCCOCCOCCOCCC(=O)ON3C(=O)CCC3=O)cccc2o1] |
| 26 | 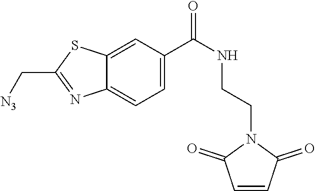 [SMILES: [N-]=[N+]=NCc1nc2ccc(C(=O)NCCN3C(=O)C=CC3=O)cc2s1] |
| 27 | 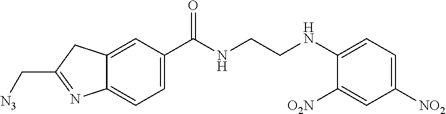 [SMILES: [N-]=[N+]=NCC1=Nc2ccc(C(=O)NCCNc3ccc([N+](=O)[O-])cc3[N+](=O)[O-])cc2C1] |
| 28 | 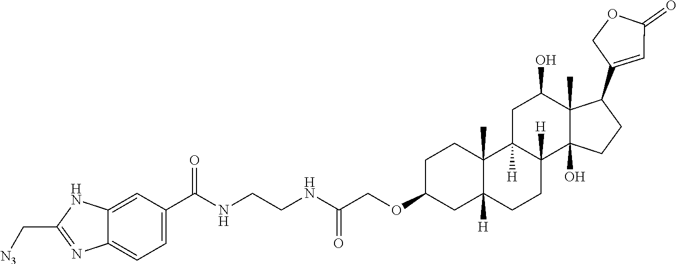 |
| 29 | 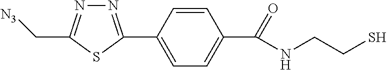 [SMILES: [N-]=[N+]=NCc1nnc(-c2ccc(C(=O)NCCS)cc2)s1] |
| 30 | 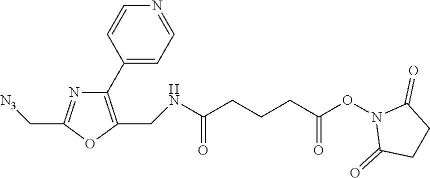 [SMILES: [N-]=[N+]=NCc1nc(-c2ccncc2)c(CNC(=O)CCCC(=O)ON2C(=O)CCC2=O)o1] |
| 31 | 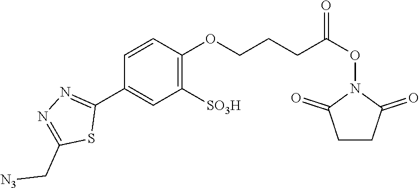 [SMILES: [N-]=[N+]=NCc1nnc(-c2ccc(OCCCC(=O)ON3C(=O)CCC3=O)c(S(=O)(=O)O)c2)s1] |
| 32 | 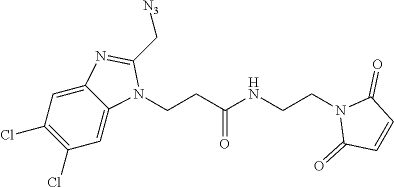 [SMILES: [N-]=[N+]=NCc1nc2cc(Cl)c(Cl)cc2n1CCC(=O)NCCN1C(=O)C=CC1=O] |
| 33 | 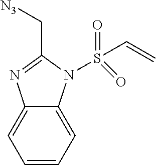 [SMILES: C=CS(=O)(=O)n1c(CN=[N+]=[N-])nc2ccccc21] |
| 34 | 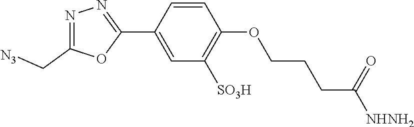 [SMILES: [N-]=[N+]=NCc1nnc(-c2ccc(OCCCC(=O)NN)c(S(=O)(=O)O)c2)o1] |
| 35 | 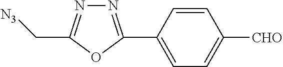 [SMILES: [N-]=[N+]=NCc1nnc(-c2ccc(C=O)cc2)o1] |
| 36 | 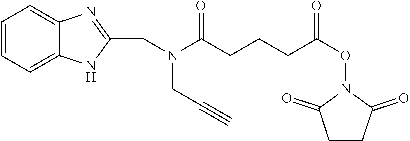 [SMILES: C#CCN(Cc1nc2ccccc2[nH]1)C(=O)CCCC(=O)ON1C(=O)CCC1=O] |
| 37 | 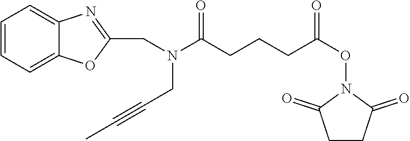 [SMILES: CC#CCN(Cc1nc2ccccc2o1)C(=O)CCCC(=O)ON1C(=O)CCC1=O] |
| 38 | 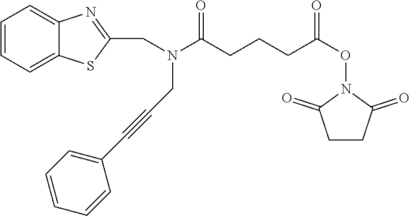 [SMILES: O=C(CCCC(=O)N(CC#Cc1ccccc1)Cc1nc2ccccc2s1)ON1C(=O)CCC1=O] |
| 39 | 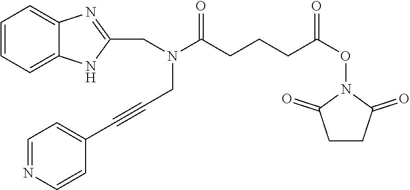 [SMILES: O=C(CCCC(=O)N(CC#Cc1ccncc1)Cc1nc2ccccc2[nH]1)ON1C(=O)CCC1=O] |
| 40 | 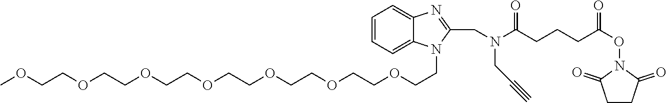 [SMILES: C#CCN(Cc1nc2ccccc2n1CCOCCOCCOCCOCCOCCOCCOC)C(=O)CCCC(=O)ON1C(=O)CCC1=O] |
| 41 | 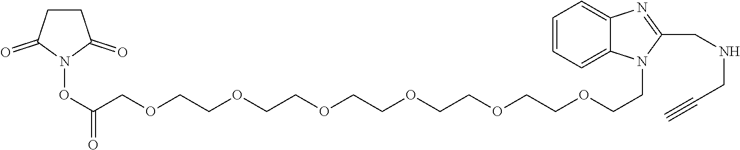 [SMILES: C#CCNCc1nc2ccccc2n1CCOCCOCCOCCOCCOCCOCC(=O)ON1C(=O)CCC1=O] |
| 42 | 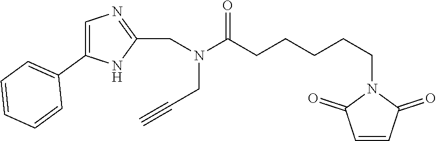 [SMILES: C#CCN(Cc1ncc(-c2ccccc2)[nH]1)C(=O)CCCCCN1C(=O)C=CC1=O] |
| 43 | 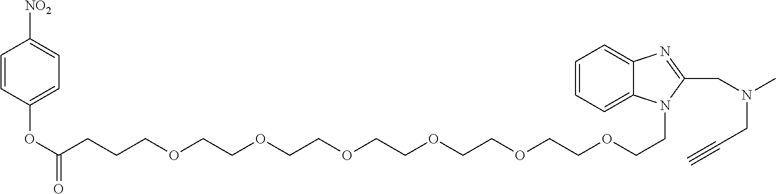 [SMILES: C#CCN(C)Cc1nc2ccccc2n1CCOCCOCCOCCOCCOCCOCCCC(=O)Oc1ccc([N+](=O)[O-])cc1] |
| 44 | 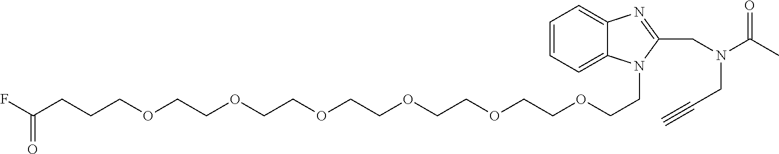 [SMILES: C#CCN(Cc1nc2ccccc2n1CCOCCOCCOCCOCCOCCOCCCC(=O)F)C(C)=O] |
| 45 | 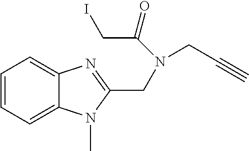 [SMILES: C#CCN(Cc1nc2ccccc2n1C)C(=O)CI] |
| 46 | 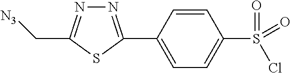 [SMILES: [N-]=[N+]=NCc1nnc(-c2ccc(S(=O)(=O)Cl)cc2)s1] |
| 47 | 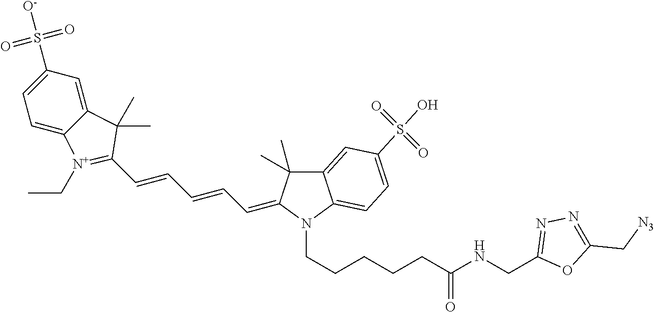 [SMILES: CC[N+]1=C(/C=C/C=C/C=C2/N(CCCCCC(=O)NCc3nnc(CN=[N+]=[N-])o3)c3ccc(S(=O)(=O)O)cc3C2(C)C)C(C)(C)c2cc(S(=O)(=O)[O-])ccc21] |
| 48 | 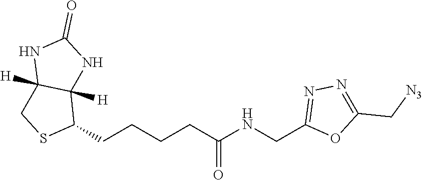 [SMILES: [N-]=[N+]=NCc1nnc(CNC(=O)CCCC[C@@H]2SC[C@@H]3NC(=O)N[C@@H]32)o1] |
| 49 | 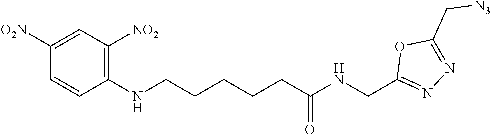 [SMILES: [N-]=[N+]=NCc1nnc(CNC(=O)CCCCCNc2ccc([N+](=O)[O-])cc2[N+](=O)[O-])o1] |
| 50 | 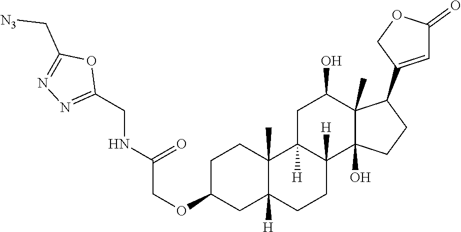 |
| 51 | 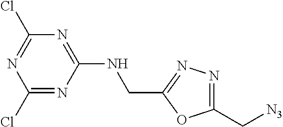 [SMILES: [N-]=[N+]=NCc1nnc(CNc2nc(Cl)nc(Cl)n2)o1] |
| 52 | 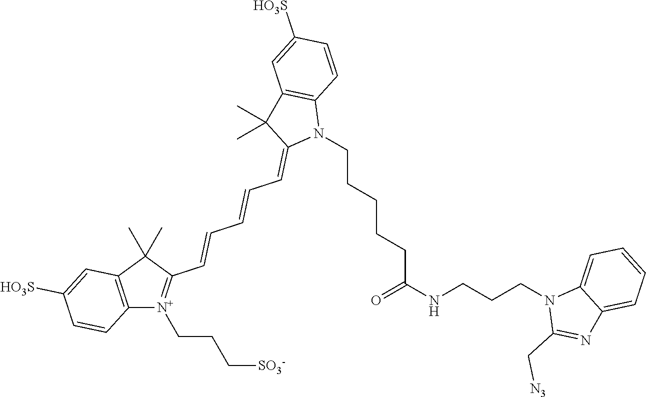 |
| 53 | 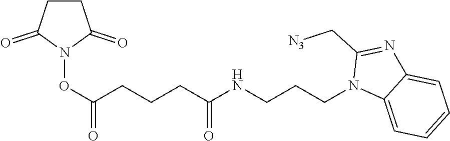 [SMILES: [N-]=[N+]=NCc1nc2ccccc2n1CCCNC(=O)CCCC(=O)ON1C(=O)CCC1=O] |
| 54 | 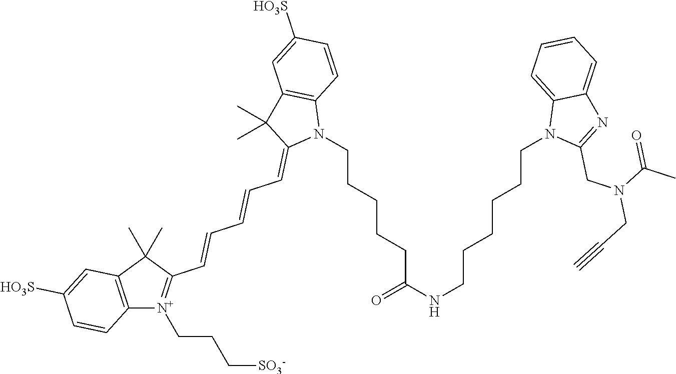 |
| 55 | 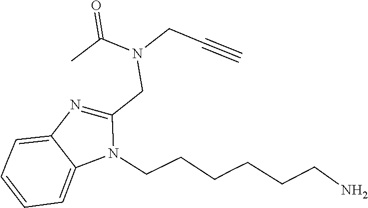 [SMILES: C#CCN(Cc1nc2c(n1CCCCCCCN)=CC=CCC=2)C(C)=O] |

### EXAMPLES

Examples of some synthetic strategies for selected compounds of the disclosure, as well as their characterization, synthetic precursors, conjugates and methods of use are provided in the examples below. Further modifications and permutations will be obvious to one skilled in the art. The examples below are given so as to illustrate the practice of this disclosure. They are not intended to limit or define the entire scope of this disclosure. It is to be understood that this disclosure is not limited to particular aspects described, as such may vary. It is also to be understood that the terminology used herein is for the purpose of describing particular aspects only and is not intended to be limiting since the scope of the present disclosure will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, representative illustrative methods and materials are now described.

Synthesis of the reactive compounds of the disclosure depends on initial preparation of certain key intermediates as illustrated in the following examples. For simplicity, all but a few of the possible substituents are shown as hydrogen. These basic structures are optionally further substituted, during or after synthesis, to give the corresponding compound conjugate substituents as defined above. It is recognized that there are many possible variations that may yield equivalent results.

The methods for the synthesis of compounds that contain a variety of reactive functional groups such as those described in Table 2 are well documented in the art. Particularly useful are amine-reactive compounds such as “activated esters” of carboxylic acids, which are typically synthesized by coupling a carboxylic acid to a relatively acidic “leaving group”. Other preferred amine-reactive groups include sulfonyl halides, which are prepared from sulfonic acids using a halogenating agent such as PCl₅ or POCl₃; halotriazines, which are prepared by the reaction of cyanuric halides with amines; and isocyanates or isothiocyanates, which are prepared from amines and phosgene or thiophosgene, respectively.

Compounds containing amines and hydrazides are particularly useful for conjugation to carboxylic acids, aldehydes and ketones. Most often these are synthesized by reaction of an activated ester of a carboxylic acid or a sulfonyl halide with a diamine, such as cadaverine, or with a hydrazine. Alternatively, aromatic amines are commonly synthesized by chemical reduction of a nitroaromatic compound. Amines and hydrazines are particularly useful precursors for synthesis of thiol-reactive haloacetamides or maleimides by standard methods.

In one aspect of the disclosure, the compounds of the disclosure are used to directly stain or label a sample so that the sample can be identified or quantitated. For instance, such compounds may be added as part of an assay for a biological target analyte, as a detectable tracer element in a biological or non-biological fluid; or for such purposes as photodynamic therapy of tumors, in which a compound conjugated sample is irradiated to selectively destroy tumor cells and tissues; or to photoablate arterial plaque or cells, usually through the photosensitized production of singlet oxygen. In one preferred embodiment, compound conjugate is used to stain a sample that comprises a ligand for which the conjugated biological substrate is a complementary member of a specific binding pair (e.g. Table 4).

Typically, the sample is obtained directly from a liquid source or as a wash from a solid material (organic or inorganic) or a growth medium in which cells have been introduced for culturing, or a buffer solution in which cells have been placed for evaluation. Where the sample comprises cells, the cells are optionally single cells, including microorganisms, or multiple cells associated with other cells in two or three dimensional layers, including multicellular organisms, embryos, tissues, biopsies, filaments, biofilms, etc.

Alternatively, the sample is a solid, optionally a smear or scrape or a retentate removed from a liquid or vapor by filtration. In one aspect of the disclosure, the sample is obtained from a biological fluid, including separated or unfiltered biological fluids such as urine, cerebrospinal fluid, blood, lymph fluids, tissue homogenate, interstitial fluid, cell extracts, mucus, saliva, sputum, stool, physiological secretions or other similar fluids. Alternatively, the sample is obtained from an environmental source such as soil, water, or air; or from an industrial source such as taken from a waste stream, a water source, a supply line, or a production lot.

TABLE 4
Representative specific binding pairs

| | |
|---|---|
| Antigen | Antibody |
| Biotin | Anti-biotin or avidin or streptavidin or neutravidin |
| IgG* | Protein A or protein G or anti-IgG antibody |
| Drug | Drug receptor |
| Toxin | Toxin receptor |
| Carbohydrate | Lectin or carbohydrate receptor |
| Peptide | Peptide receptor |
| Nucleotide | Complimentary nucleotide |
| Protein | Protein receptor |
| Enzyme substrate | Enzyme |
| DNA (RNA) | aDNA (aRNA)** |
| Hormone | Hormone receptor |
| Psoralen | Nucleic acid |
| Target molecule | RNA or DNA aptamer |
| Ion | Ion chelator |

*IgG is an immunoglobulin;
**aDNA and aRNA are the antisense (complementary) strands used for hybridization

### Example 1. The Preparation of Compound 1

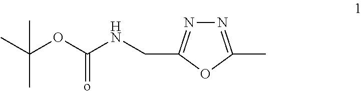

(5-Methyl-1,3,4-oxadiazol-2-yl)methanamine (10 g) and di-tert-butyl dicarbonate (20 g) are dissolved in dioxane (100 mL) room temperature. To the solution is added triethylamine (15 mL) room temperature. The reaction mixture is stirred at room temperature for 24 hours and concentrated under a high vacuum. The residue is dissolved in ethyl acetate (200 mL) and washed with 1% HCl (2×200 mL) and water (2×200 mL) respectively. The ethyl acetate solution is dried over anhydrous sodium sulfate, filtered and concentrated. The residue is purified by flash chromatography (ethyl acetate/dichloromethane, 0-10%) to give compound 2 as a white solid.

### Example 2. The Preparation of Compound 2

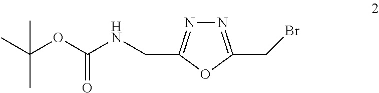 [SMILES: CC(C)(C)OC(=O)NCc1nnc(CBr)o1]

Compound 1 (5 g) is dissolved in CCl₄ (100 ml) and NBS (26 g) followed by the addition of benzoyl peroxide (800 mg). The reaction is heated to reflux and stirred for 48 hours, after which time the mixture is cooled to room temperature and filtered. The filtrate is concentrated and purified on a silica gel flash column chromatography (EtOAc/Hexanes, 0% to 30%) to give the product as a white solid.

### Example 3. The Preparation of Compound 3

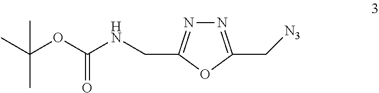 [SMILES: CC(C)(C)OC(=O)NCc1nnc(CN=[N+]=[N-])o1]

Compound 2 (1.03 g) is dissolved in N,N-dimethylformamide (DMF, 30 mL). To the DMF solution, sodium azide (4×0.5 g) is added in ice bath (in 4 portions) and the reaction mixture is stirred at room temperature. The reaction mixture is stirred at room temperature until complete conversion to the desired product. The mixture is poured into water (500 mL) while stirring. The white precipitate is collected, washed with water, and air-dried to give compound 3 as a white solid.

### Example 4. The Preparation of Compound 4

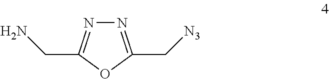 [SMILES: [N-]=[N+]=NCc1nnc(CN)o1]

Compound 3 (3 g) is dissolved in dichloromethane (20 mL). To the solution is added 25% trifluoroacetic acid/dichloromethane solution (20 mL). The reaction mixture is stirred at room temperature for 6 hours and concentrated under a high vacuum. The residue is purified on a C18 reverse phase silica gel column by HPLC to give the desired Compound 4 as an off-white solid.

### Example 5. The Preparation of Compound 5

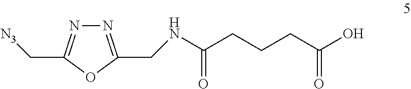 [SMILES: [N-]=[N+]=NCc1nnc(CNC(=O)CCCC(=O)O)o1]

Compound 4 (1 g) is dissolved in DMF (10 mL) followed by adding dihydro-2H-pyran-2,6(3H)-dione (0.9 g) and triethylamine (1 mL). The mixture is stirred at room temperature for 24 hours and concentrated. The residue is poured into water (100 mL), and the precipitate is collected and air-dried. The crude solid is purified on a C18 reverse phase silica gel column by HPLC to give the desired Compound 5 as an off-white solid.

### Example 6. The Preparation of Compound 6

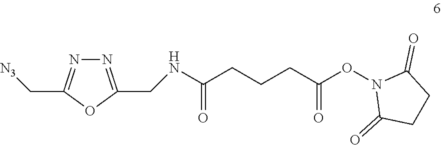 [SMILES: [N-]=[N+]=NCc1nnc(CNC(=O)CCCC(=O)ON2C(=O)CCC2=O)o1]

To the solution of compound 5 (100 mg) in DMF (2 mL), Et₃N (0.32 mL) and N,N,N′,N′-tetramethyl-O—(N-succinimidyl)uronium tetrafluoroborate (130 mg) are added. The reaction mixture is stirred at room temperature for 30-60 minutes and poured into ether (50 mL). The precipitate is collected by filtration and washed with ether (50 mL) and dried to give Compound 6 as an off-white solid.

### Example 7. The Preparation of Compound 7

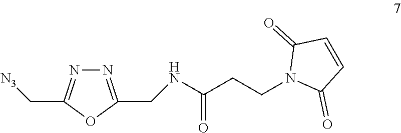 [SMILES: [N-]=[N+]=NCc1nnc(CNC(=O)CCN2C(=O)C=CC2=O)o1]

To the solution of Compound 4 (100 mg) in DMF (5 mL) is added 3-Maleimidopropionic acid N-hydroxysuccinimide ester (200 mg), followed with the addition of triethylamine (0.2 mL). The reaction mixture is stirred at room temperature for 12 hours. The mixture is concentrated, and the residue is poured into water. The suspension is filtered to collect the precipitate that is air-dried. The crude solid is purified by flash chromatography (DCM-MeOH, 0-10%) to give Compound 7 as an off-white solid.

### Example 8. The Preparation of Compound 8

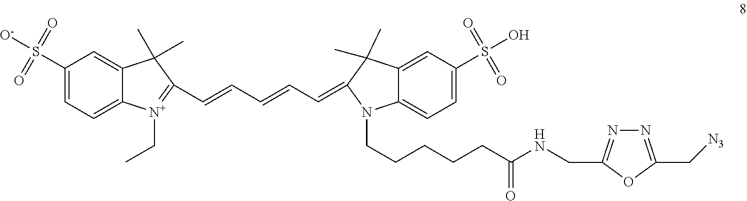 [SMILES: CC[N+]1=C(/C=C/C=C/C=C2/N(CCCCCC(=O)NCc3nnc(CN=[N+]=[N-])o3)c3ccc(S(=O)(=O)O)cc3C2(C)C)C(C)(C)c2cc(S(=O)(=O)[O-])ccc21]

To the solution of Compound 4 (50 mg) in DMF (5 mL) is added Cy5 NHS ester, followed with the addition of triethylamine (0.1 mL). The reaction mixture is stirred at room temperature for 12 hours. The mixture is concentrated, and the residue is purified on a C18 reverse phase silica gel column by HPLC to give the desired Compound 8 as a blue solid.

### Example 9. The Preparation of Compound 9

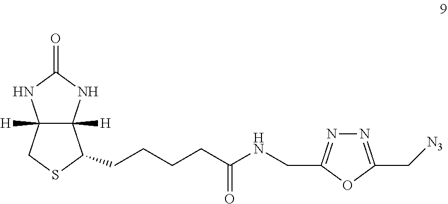 [SMILES: [N-]=[N+]=NCc1nnc(CNC(=O)CCCC[C@@H]2SC[C@@H]3NC(=O)N[C@@H]32)o1]

To the solution of Compound 4 (50 mg) in DMF (5 mL) is added biotin NHS ester, followed with the addition of triethylamine (0.1 mL). The reaction mixture is stirred at room temperature for 12 hours. The mixture is concentrated, and the residue is purified on a C18 reverse phase silica gel column by HPLC to give the desired Compound 9 as an off-white solid.

### Example 10. The Preparation of Compound 10

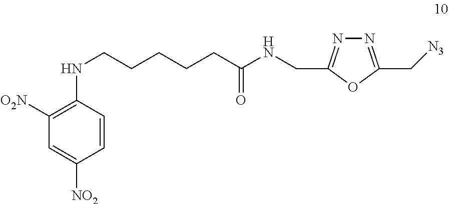 [SMILES: [N-]=[N+]=NCc1nnc(CNC(=O)CCCCCNc2ccc([N+](=O)[O-])cc2[N+](=O)[O-])o1]

To the solution of Compound 4 (50 mg) in DMF (5 mL) is added DNP-X succinimidyl ester, followed with the addition of triethylamine (0.1 mL). The reaction mixture is stirred at room temperature for 12 hours. The mixture is concentrated, and the residue is purified on a C18 reverse phase silica gel column by HPLC to give the desired Compound 10 as an off-white solid.

### Example 11. The Preparation of Compound 11

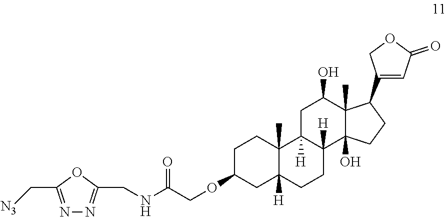

To the solution of Compound 4 (50 mg) in DMF (5 mL) is added digoxigenin succinimidyl ester, followed with triethylamine (0.1 mL). The reaction mixture is stirred at room temperature for 12 hours. The reaction mixture is stirred at room temperature for 12 hours. The mixture is concentrated, and the residue is poured into water. The suspension is filtered to collect the precipitate that is air-dried. The crude solid is purified by flash chromatography (dichloromethane-MeOH, 0-10%) to give Compound 11 as an off-white solid.

### Example 12. The Preparation of Compound 12

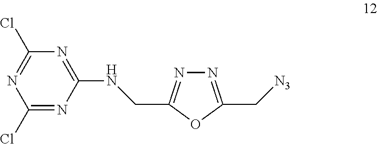 [SMILES: [N-]=[N+]=NCc1nnc(CNc2nc(Cl)nc(Cl)n2)o1]

To the solution of Compound 4 (10 mg) in tetrahydrofuran (THF, 2 ml) is added 0.1 ml THF solution of cyanuric chloride (15 mg) and 0.01 mL of triethylamine. The reaction mixture is stirred at room temperature for 3 hours and concentrated under a high vacuum to remove THF. The residue is washed with ether multiple times until most of the unreacted cyanuric chloride is removed. The residue is dissolved in dichloromethane (50 mL) and washed with water. The organic phase is evaporated to give the desired dichlorotriazine.

### Example 13. Preparation of Compound 13

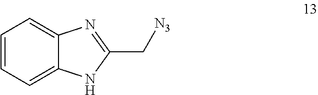 [SMILES: [N-]=[N+]=NCc1nc2ccccc2[nH]1]

To the solution of 2-(chloromethyl)benzimidazole (2 g) in anhydrous dimethyl sulfoxide (DMSO, 30 mL) is added NaN₃ (1.6 g). The resulting mixture is stirred at room temperature for 6 hours. The reaction progress is monitored by HPLC. After the reaction completion, water (100 mL) is added. The mixture is extracted with diethyl ether (50 mL×3). The combined organic extracts are washed with water (100 mL×1) and brine (100 mL×1), dried over anhydrous Na₂SO₄. The solvent is removed and dried under vacuum to yield Compound 13 as a light-yellow solid.

### Example 14. Preparation of Compound 14

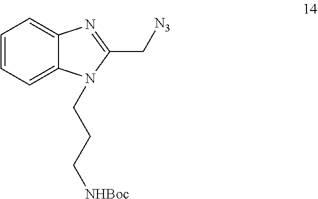 [SMILES: CC(C)(C)OC(=O)NCCCn1c(CN=[N+]=[N-])nc2ccccc21]

To the solution of Compound 13 (0.4 g) and 3-(Boc-amino)propyl bromide (1.1 g, in 40 mL of anhydrous acetonitrile) is added anhydrous K₂CO₃ (1.6 g). The resulting mixture is refluxed while stirring overnight. The reaction progress is monitored by HPLC. After the reaction completion, the mixture is filtered to remove the solid. The solvent is evaporated to yield crude Compound 14. The crude product is purified on a silica-gel column eluting with a gradient of methanol in dichloromethane from 0% to 5% to give the pure Compound 14 as a light-yellow solid.

### Example 15. Preparation of Compound 15

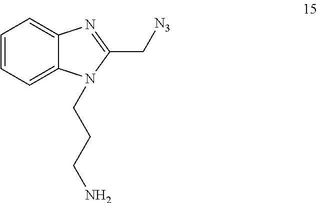 [SMILES: [N-]=[N+]=NCc1nc2ccccc2n1CCCN]

To a solution of Compound 14 (485 mg) and anisole (159 mg) in anhydrous dichloromethane (4 mL) is added TFA (2 mL) under stirring. The resulting mixture is stirred at room temperature for 2 hours. The reaction progress is monitored by HPLC. After the reaction completion, the reaction mixture is concentrated to give the crude product that is purified by reverse-phase HPLC to yield pure Compound 15 as a light-yellow solid.

### Example 16. Preparation of Compound 16

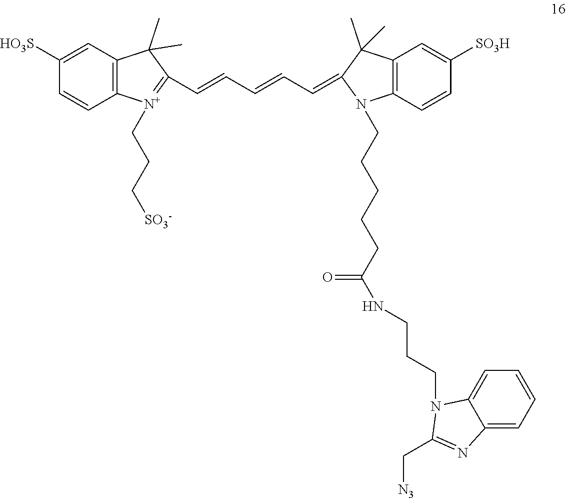

To the solution of Cy5 succinimidyl ester (25 mg) and TEA (7.3 mg, 0.01 mL) in anhydrous DMF (2 mL) is added Compound 15 (9 mg/0.5 mL of anhydrous DMF) in portions. The reaction progress is monitored by HPLC. After the reaction completion, the reaction mixture is concentrated to give the crude product that is purified by reverse-phase HPLC to yield pure Compound 16 as a blue solid.

### Example 17. Preparation of Compound 17

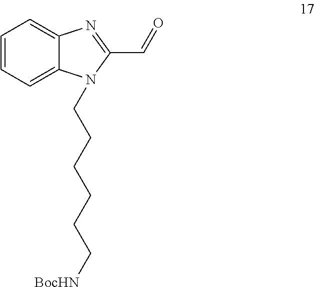 [SMILES: CC(C)(C)OC(=O)NCCCCCCn1c(C=O)nc2ccccc21]

To 1H-benzo[d]imidazole-2-carbaldehyde (320 mg) and tert-Butyl (6-bromohexyl)carbamate (1.12 g/50 mL of anhydrous DMSO) is added anhydrous K₂CO₃ (1.3 g). The reaction mixture is heated at 40° C. for 2 hours. Water (100 mL) is added, and the mixture is extracted with ethyl acetate (100 mL×3). The combined organic extracts are washed with brine (100 mL×1), dried over anhydrous Na₂SO₄, and concentrated to give the crude product that is purified on a silica-gel column, eluting with a gradient of ethyl acetate in dichloromethane from 0% to 15% to give pure Compound 17 as a light-yellow oil.

### Example 18. Preparation of Compound 18

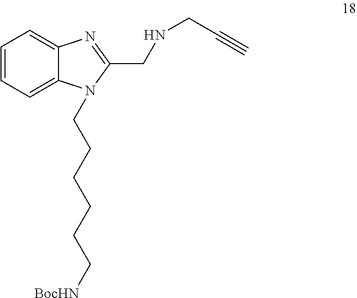 [SMILES: C#CCNCc1nc2ccccc2n1CCCCCCNC(=O)OC(C)(C)C]

Compound 17 (150 mg) is dissolved in anhydrous dichloromethane (10 mL) and cooled to 0-4° C. in an ice-water bath. To the above solution is added propargylamine (60 mg) under stirring. The resulting mixture is stirred at 0-4° C. for 30 min. NaBH(Oac)₃ (230 mg) is added to the reaction mixture in portions while stirring. The reaction mixture is further stirred at room temperature for 6 hours. The reaction progress is monitored by HPLC. Upon the reaction completion, the solvent and propargylamine are removed under a high vacuum. The crude Compound 18 is dried and directly used for the next-step reaction without further purification.

### Example 19. Preparation of Compound 19

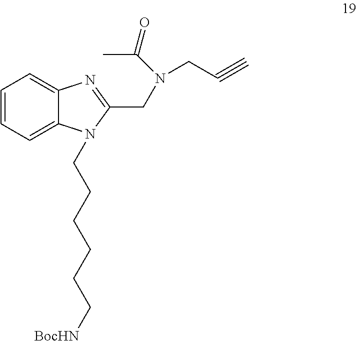 [SMILES: C#CCN(Cc1nc2ccccc2n1CCCCCCNC(=O)OC(C)(C)C)C(C)=O]

To Compound 18 (166 mg) in anhydrous DMF (1 mL) is added acetic anhydride (110 mg, 0.1 mL) and TEA (220 mg, 0.3 mL). The resulting mixture is stirred at room temperature for 30 min. The reaction progress is monitored by analytical HPLC. Upon the reaction completion, the reaction mixture is concentrated and dried under high vacuum to yield Compound 19 as a light-yellow solid.

### Example 20. Preparation of Compound 20

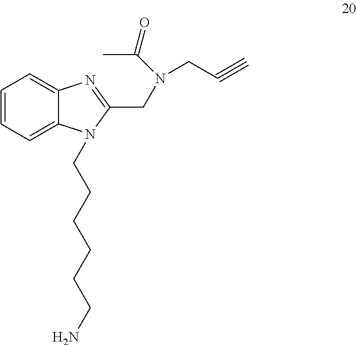 [SMILES: C#CCN(Cc1nc2ccccc2n1CCCCCCN)C(C)=O]

To Compound 19 (185 mg) and anisole (47 mg) in anhydrous dichloromethane (3 mL) is added TFA (1.5 mL) under stirring at 0-4° C. The resulting mixture is stirred at room temperature for 1 hour. The reaction progress is monitored by HPLC. After the reaction completion, the reaction mixture is concentrated and dried under a high vacuum to yield the crude product. The crude product is purified by reverse-phase HPLC to yield the pure Compound 20 as a light-yellow solid.

### Example 21. Preparation of Compound 21

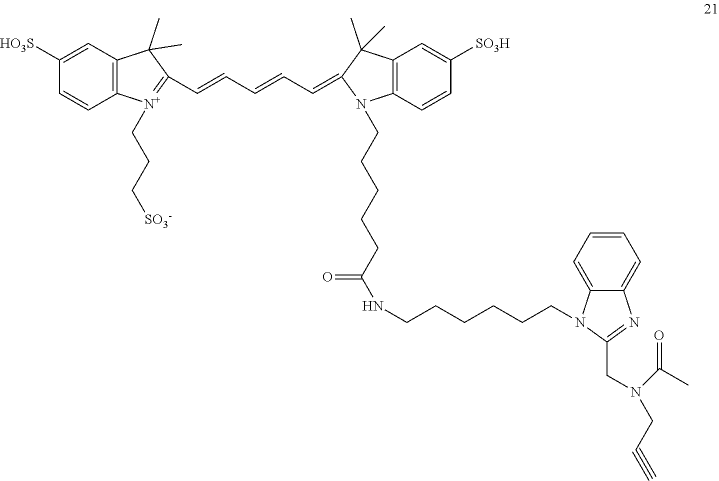

To 2 mL of the anhydrous DMF solution of Cy5 succinimidyl ester (30 mg) and TEA (11.6 mg, 0.016 mL) is added 0.5 mL of anhydrous DMF solution of Compound 20 (15 mg) in portions. The reaction progress is monitored by analytical HPLC. After the reaction completion, the reaction mixture is concentrated and dried under a high vacuum to yield the crude product. The crude product is purified by reverse-phase HPLC to yield pure Compound 21 as a blue solid.

### Example 22. The Preparation of Compound 22

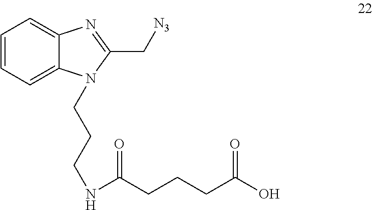 [SMILES: [N-]=[N+]=NCc1nc2ccccc2n1CCCNC(=O)CCCC(=O)O]

Compound 22 is analogously prepared from the reaction of Compound 15 with dihydro-2H-pyran-2,6(3H)-dione as described in Example 5.

### Example 23. The Preparation of Compound 23

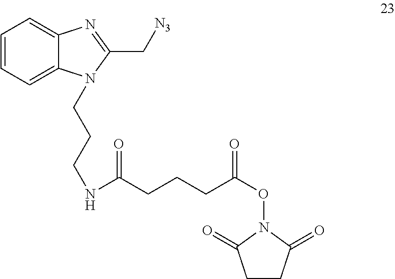 [SMILES: [N-]=[N+]=NCc1nc2ccccc2n1CCCNC(=O)CCCC(=O)ON1C(=O)CCC1=O]

Compound 23 is analogously prepared from the reaction of Compound 22 with N,N,N′,N′-tetramethyl-O—(N-succinimidyl)uronium tetrafluoroborate (130 mg) as described in Example 6.

### Example 24. The Click Reactions of Azido Compounds at 0.2 mM

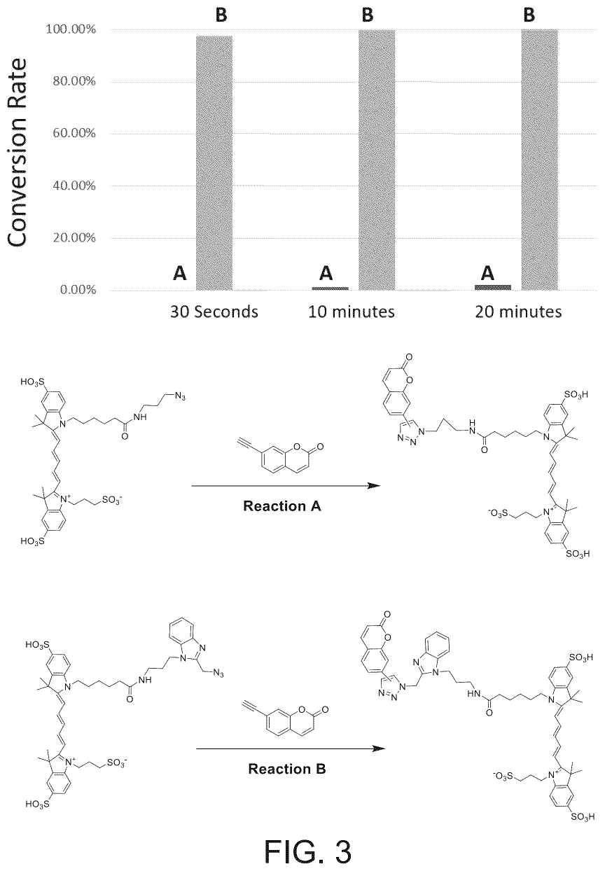

An azido compound is dissolved in DMF to make 2 mM azido compound stock solution. 2 mM DMF stock solution of an alkyne is similarly prepared. To 0.6 mL of 100 mM reaction buffer consisting of HEPES buffer (pH 7.5) and DMF (75/25, v/v) are added 0.1 ml of azido compound stock solution, 0.1 mL of the alkyne stock solution, and 0.1 mL of 2 mM CuSO₄ aqueous solution at room temperature with stirring. To the reaction mixture 0.1 mL of 50 mM sodium ascorbate aqueous solution is added to initiate the click reaction. Upon the addition of sodium ascorbate, the reaction is immediately monitored by HPLC. The results are summarized in FIG. 3.

### Example 25. The Click Reactions of Azido Compounds at 0.02 mM

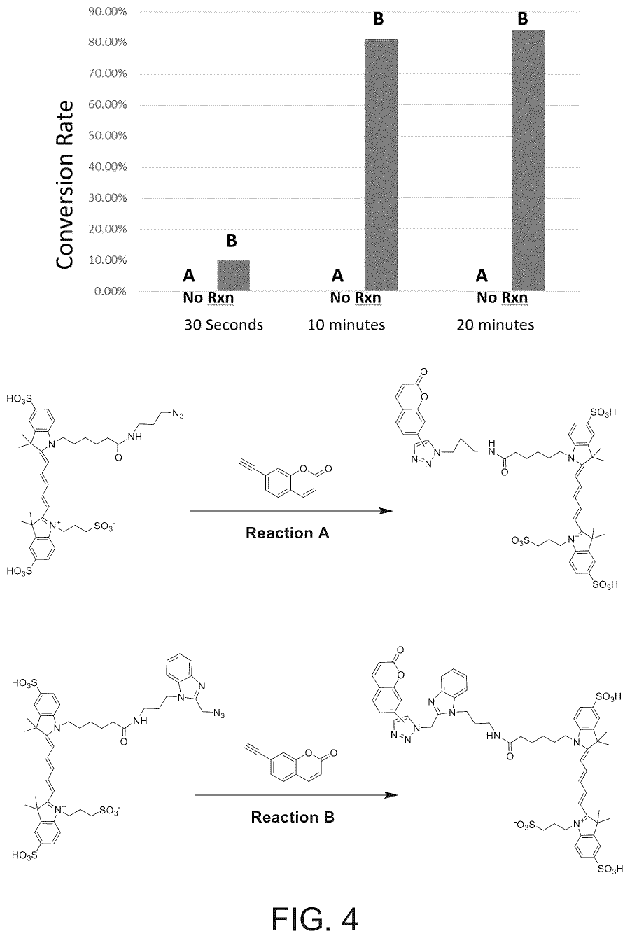

An azido compound is dissolved in DMF to make 0.2 mM azido compound stock solution. DMF stock solution of an alkyne is similarly prepared. To 0.6 mL of 100 mM reaction buffer consisting of HEPES buffer (pH 7.5) and DMF (75/25, v/v) are added 0.1 ml of the azido compound stock solution, 0.1 mL of the alkyne stock solution, and 0.1 mL of 0.2 mM CuSO₄ aqueous solution at room temperature with stirring. To the reaction mixture 0.1 mL of 5 mM sodium ascorbate aqueous solution is added to initiate the click reaction. Upon the addition of sodium ascorbate, the reaction is immediately monitored by HPLC. The results are summarized in FIG. 4.

### Example 26. The Click Reactions of Alkyne Compounds at 0.2 mM

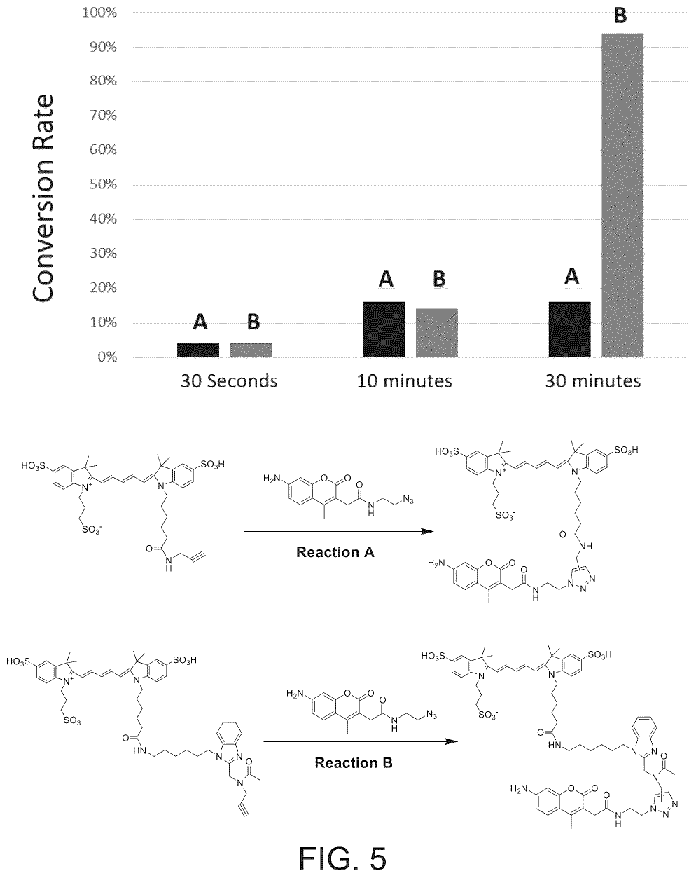

An alkyne is dissolved in DMF to make 2 mM alkyne stock solution. 2 mM DMF stock solution of an azido compound is similarly prepared. To 0.6 mL of 100 mM reaction buffer consisting of HEPES buffer (pH 7.5) and DMF (75/25, v/v) are added 0.1 ml of the alkyne stock solution, 0.1 mL of the azido compound stock solution, and 0.1 mL of 2 mM CuSO₄ aqueous solution at room temperature with stirring. To the reaction mixture 0.1 mL of 50 mM sodium ascorbate aqueous solution is added to initiate the click reaction. Upon the addition of sodium ascorbate, the reaction is immediately monitored by HPLC. The results are summarized in FIG. 5.

### Example 27. The Click Reactions of Alkyne Compounds at 0.02 mM

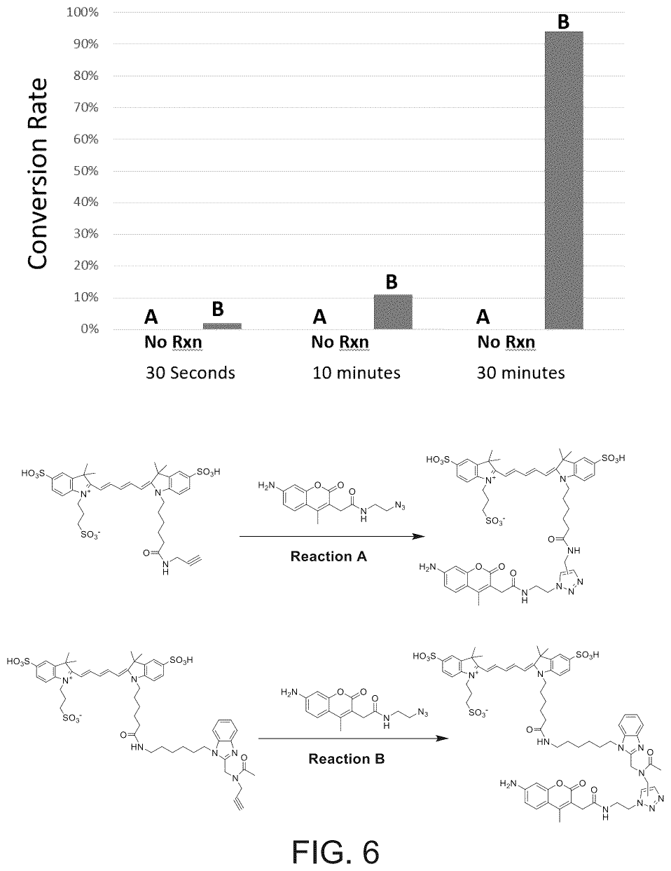

An alkyne is dissolved in DMF to make 0.2 mM alkyne stock solution. 0.2 mM DMF stock solution of an azido compound is similarly prepared. To 0.6 mL of 100 mM reaction buffer consisting of HEPES buffer (pH 7.5) and DMF (75/25, v/v) are added 0.1 ml of the alkyne stock solution, 0.1 mL of the azido compound stock solution, and 0.1 mL of 0.2 mM CuSO₄ aqueous solution at room temperature with stirring. To the reaction mixture 0.1 mL of 5 mM sodium ascorbate aqueous solution is added to initiate the click reaction. Upon the addition of sodium ascorbate, the reaction is immediately monitored by HPLC. The results are summarized in FIG. 6.

### Example 28. Preparation of Goat Anti-Mouse IgG Conjugates

Goat Anti-Mouse IgG (GAM) is dissolved in 10 mM NaHCO₃(pH 8.2) buffer to make a 5 mg/mL solution. To the aqueous GAM protein solution is added the DMF solution of Compound 23 (20 equivalents). The solution is rotated at room temperature for 3 hours and the reaction mixture is transferred to an Amicon Ultra filter (MWCO=10 kDa) to remove DMF. The protein is recovered into the initial volume with PBS buffer.

Cation exchange chromatograph is used to remove the free compound. Conjugation mixture is loaded to UNOsphere™ S resin (Bio-Rad) in low salt buffer [50 mM MES Buffer (pH=5.0)], and incubated at room temperature for 10 minutes, repeatedly loading the sample for 3 times to get the maximum binding. After loading, the medium is washed with low salt buffer to the baseline to remove all the non-conjugated compounds. The retained Compound 23-modified GAM conjugate on the cation exchange resin is released by elevating both the pH and ionic strength with high salt phosphate buffer [10 mM phosphate buffer (pH=7.4)+1.OM NaCl buffer/methanol, 90/10]. Protein A and Protein G affinity resins can also be used to remove the free compound with comparable results. A HiTrap Protein G HP 1 mL column (GE Lifesciences) is pre-equilibrated with 10 mM Phosphate buffer, pH 7.4, and the SEC-purified product is slowly injected at <1 mg/mL and allowed to incubate for 30 minutes to bind. The column is washed with >10 column volumes of 10 mM Phosphate buffer to wash unbound compound material off while monitoring absorption of the eluate at 280 nm and 414 nm to ensure all excess material is removed. The conjugate is eluted by washing the column with 4 column volumes of 0.1 M Glycine pH 2.3. The eluted fractions are combined and pH-adjusted back to neutral using 1 M Tris pH 8. After the free compound is removed, the conjugate solution is concentrated with Amicon Ultra Filter (MWCO=30 kD) and loaded to a size exclusion column (Superdex 200, GE life sciences) to separate conjugate and unconjugated antibody. The column is equilibrated with PBS buffer, and the conjugated compound-antibody conjugate is eluted before free antibodies. For effective labeling, the degree of substitution should fall between 5-15 moles of conjugated compound dye to one mole of antibody for most antibodies. As is well known in the art, the optima DOS depends on the properties of antibody to be labeled. The optimal labeling DOS is determined empirically by preparing a series of dye-conjugates over a range of DOS and comparing the desired signal/background. In some cases, a higher DOS may provide bright signal while in other cases higher DOS could reduce the affinity of the antibody to be labeled. Other Compound 23-labeled antibody conjugates (e.g., Compound 23-labeled CD45 conjugate) can be analogously prepared.

The Compound 23-labeled antibody conjugates might be reacted with an alkyne compound to make tagged antibody conjugates. For example, Compound 23-labeled antibody can be conjugated to an alkyne-modified enzyme (such as HRP) to make an enzyme-antibody conjugate. Similarly, Compound 23-labeled antibody might be conjugated to an alkyne-Qdot to make an antibody-Qdot conjugates.

### Example 29. The Labeling and Staining of Cells with Compound 16

Hela cells is plated in a 96-well plate overnight in growth medium at 10,000 to 40,000 cells/well/100 μL. The cells are incubated with 0.01 mM 5-ethynyl uridine for 3 hours to label the cells with an alkyne. After incubation, the cell medium is removed. To each well of cells is add 0.1 mL ice-cold 90% methanol in PBS and incubated for 15 minutes at room temperature to fix the cells. The fixation buffer is then removed, and the cells are washed twice with PBS. To each well of cells 2 mM Compound 16 stock solution (0.05 mL), 2 mM CuSO₄ aqueous solution (0.05 mL), and 5 mM sodium ascorbate aqueous solution (0.05 mL) are added sequentially. The cells are incubated for 30 minutes and washed with PBS three times. The cells are imaged with a Cy5 filter set. Hoechst 33342 is used to stain nuclei using a literature procedure (See AssayWise Letters 2017, 6).

For primary incubation, cells are incubated with a primary conjugate specific to an antigen of interest, negative cells served as a negative non-specific binding reference. A control population or an established commercial conjugate is used as a positive control. Primary antibody-compounds are incubated at various concentrations with volume dilutions typically from 10 nM-500 μM for 30 minutes.

For secondary antibody labeling, an unlabeled primary antibody to the antigen of interest is incubated at 1-50 μg/mL, or another titrated amount. Following primary incubation, cells are rinsed with 5 volumes staining buffer and spun down for 3-5 minutes. Species reactive secondary conjugated compounds are incubated at concentrations with volume dilutions from 10-500 nM for 30-60 minutes. Following secondary incubation, cells are rinsed with 3-5 volumes staining buffer and spun down for 3-5 minutes. Cells are resuspended for testing in DPBS+0.2% BSA, 0.05% sodium azide.

For streptavidin-compound conjugate labeling, cells are incubated with a biotinylated primary antibody to the marker of interest, as detailed above for the secondary antibody labeling, instead of an unlabeled primary. Following the primary washing, cells are resuspended and incubated with streptavidin-compounds at 1-100 nM volume dilutions for 30 minutes. Following secondary incubation, cells are rinsed with 5 volumes staining buffer and spun down for 3-5 minutes. Cells are resuspended for testing. If further signal amplification is desired, cells could be incubated with an unlabeled primary antibody and then subsequently followed with a species reactive biotinylated secondary antibody prior to incubation with streptavidin conjugates.

It will be understood that the particular antibody conjugate used and the specific reaction components and particular reaction conditions used can have an effect on the results obtained. Routine experimentation should be carried out to determine preferred reaction components, such as buffers or lyse solutions, and reaction conditions, including staining times and temperatures. Such routine optimization of assay conditions is standard practice in the field of immunostaining-based assays.

What is claimed is:

1. A compound, wherein the compound is represented by Formula A or is a salt thereof: 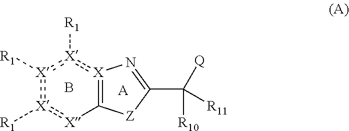
wherein
X is CR₁ or N;
Z is O, S, N—R₈, P—R₈, O═P—R₈, or O═P—OR₈;
Q is N₃, 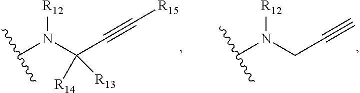
alkynyl or substituted alkynyl;
X″ is R₂ or X′—R₁;
each X′ is independently absent or X,
wherein, when present, 1 to 4 X′ together form Ring B, fused to Ring A;
each of R₁ and R₂ is independently selected from H, halogen, FG, BDM, L-FG, L-BDM, L, L-BS, FD, L-FD, aliphatic, heteroaliphatic, acyl, allyl, allenyl, propargyl, carboxy, sulfonate, PEG, WSG, alkoxy, aryloxy, alkylthiol, arylthiol, nitro, nitroso, cyano, amino, hydroxy, phosphonyl, sulfonyl, carbonyl, boronic acid, aryl, heteroaryl, and optionally substituted versions thereof;
each of R₈, R₁₀, and R₁₁ is independently selected from H, alkyl, heteroalkyl, FG, BDM, L-FG, L-BDM, L, L-BS, FD, L-FD, acyl, sulfonyl, aryl, heteroaryl and optionally substituted versions thereof;
each of R₁₂, R₁₃, R₁₄, and R₁₅ is selected from independently H, alkyl, FG, BDM, L-FG, L-BDM, L, L-BS, FD, L-FD, acyl, sulfonyl, aryl, heteroaryl, and optionally substituted versions thereof.

2. The compound of claim 1, wherein the compound of Formula A is represented by Formula I or is a salt thereof: 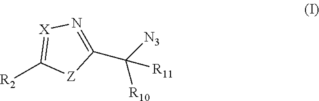
wherein
X is CR₁ or N;
Z is O, S, N—R₈, P—R₈, O═P—R₈, or O═P—OR₈;
each of R₁ and R₂ is independently selected from H, halogen, FG, BDM, L-FG, L-BDM, L, L-BS, FD, L-FD, aliphatic, heteroaliphatic, carboxy, sulfonate, PEG, WSG, alkoxy, aryloxy, alkylthiol, arylthiol, nitro, nitroso, cyano, amino, hydroxy, phosphonyl, sulfonyl, carbonyl, boronic acid, aryl, heteroaryl and optionally substituted versions thereof;
each of R₈, R₁₀, and R₁₁ is independently selected from H, alkyl, FG, BDM, L-FG, L-BDM, L, L-BS, FD, L-FD, acyl, sulfonyl, aryl, heteroaryl, and optionally substituted versions thereof;

3. The compound of claim 2, wherein
X is N;
Z is O, S, or N—R₈;
R₂ is selected from H, or an optionally substituted carboxy, sulfonate, PEG, alkoxy, aryloxy, aryl, and heteroaryl;
each of R₈, R₁₀ and R₁₁ is independently H or an optionally substituted alkyl, alkylamino, aryl, and heteroaryl.

4. The compound of claim 3, wherein
X is N;
Z is O, S, or N—R₈;
R₂ is selected from

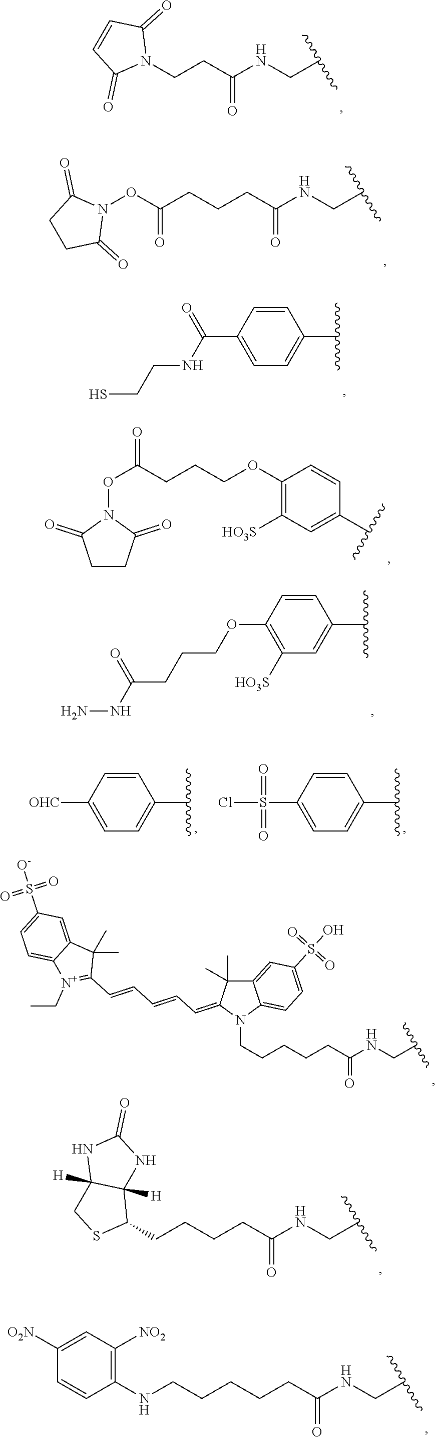

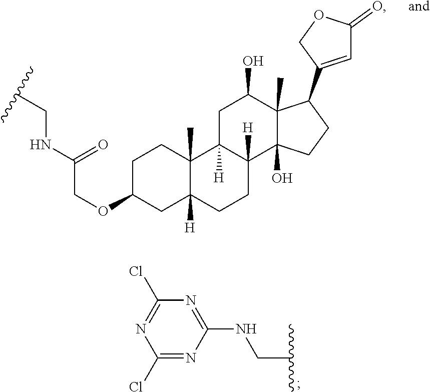

5. The compound of claim 1, wherein
X is CR₁;
Z is O, S, or N—R₈;
each of R₁ and R₂ is independently selected from 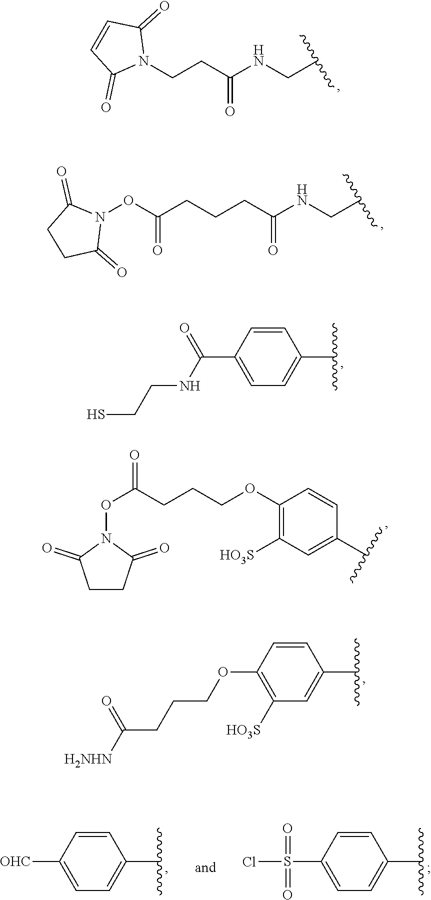
each of R₈, R₁₀, and R₁₁ is independently selected from H or an optionally substituted alkyl, aryl, and heteroaryl.

6. The compound of claim 1, wherein at least one of R₁, R₂, R₈, R₁₀, or R₁₁ is or comprises FD or L-FD.

7. The compound of claim 6, wherein FD is selected from coumarin, styryl, fluorescein, rhodamine, cyanine, BODIPY, phthalocyanine, porphyrin, and fluorescent protein.

8. The compound of claim 2, wherein the compound is selected from

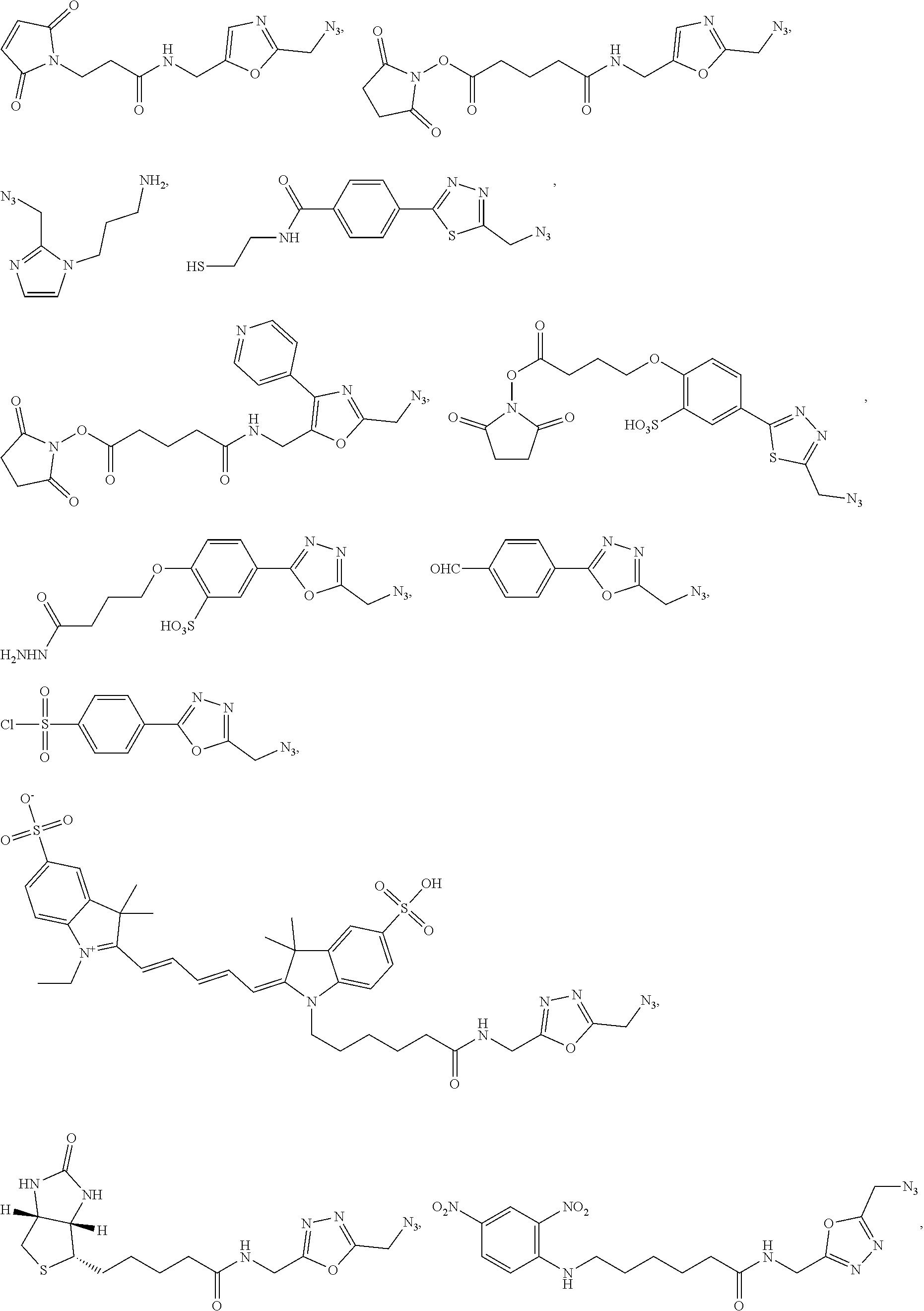

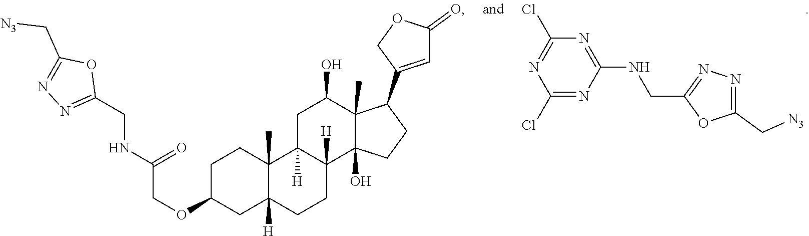

9. The compound of claim 1, wherein the compound is represented by Formula (II): 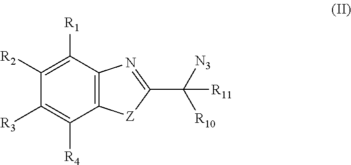
wherein
Z is O, S, N—R₈, P—R₈, O═P—R₈, or O═P—OR₈;
each of R₁, R₂, R₃, and R₄ is independently selected from H, halogen, FG, BDM, L-FG, L-BDM, L, L-BS, FD, L-FD, carboxy, sulfonate, PEG, WSG, alkoxy, aryloxy, thiol, alkylthiol, arylthiol, nitro, nitroso, cyano, amino, hydroxy, phosphonyl, sulfonyl, carbonyl, boronic acid, aryl, heteroaryl, and optionally substituted versions thereof;
each of R₈, R₁₀, and R₁₁ is independently selected from H, optionally substituted alkyl, FG, BDM, L-FG, L-BDM, L, L-BS, FD, L-FD, optionally substituted aryl, and optionally substituted heteroaryl.

10. The compound of claim 9, wherein
Z is O, S, or N—R₈;
each of R₁, R₂, R₃, and R₄ is independently selected from H, halogen, or an optionally substituted carboxy, sulfonate, PEG, alkoxy, aryloxy, aryl, and heteroaryl;
each of R₈, R₁₀, and R₁₁ is independently selected from H or an optionally substituted alkyl, aryl, and heteroaryl.

11. The compound of claim 9, wherein each of R₁, R₂, R₃, R₄, and R₈ is independently selected from H, halogen,

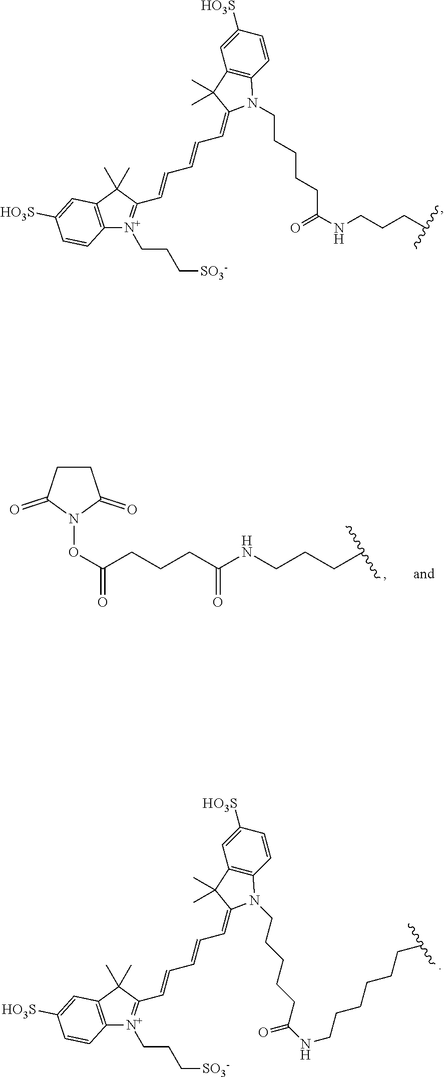

12. The compound of claim 9, wherein WSG is selected from sulfonate, PEG, carboxyalkyl, sulfonylalkyl, phosphonylalkyl, hydroxyalkyl, aminoalkyl, and ammoniumylalkyl.

13. The compound of claim 9, wherein FG is selected from an activated ester, aldehyde, maleimide, amino, hydroxylamine, and hydrazine.

14. The compound of claim 9, wherein one of R₁ to R₁₁ comprises a FD or L-FD.

15. The compound of claim 9, BS is an antibody, antigen, protein, peptide, oligonucleotide, DNA, RNA, PNA, aptamer, or a cell.

16. The compound of claim 9, wherein each of R₁, R₂, R₃, R₄, and R₈ is independently selected from H, halogen,

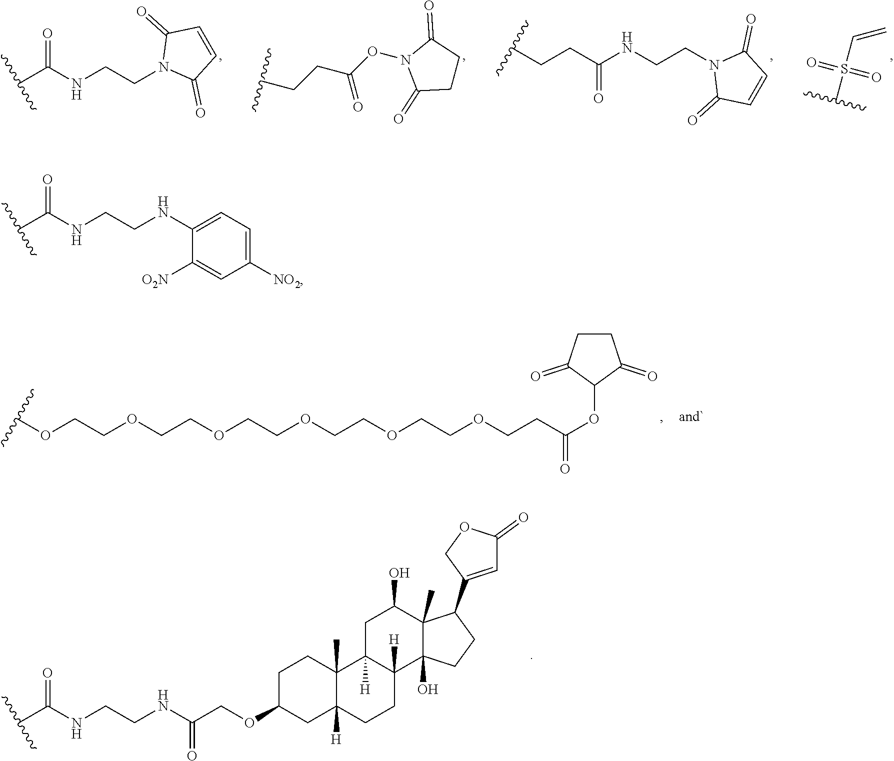

17. The compound of claim 9, wherein the compound is selected from

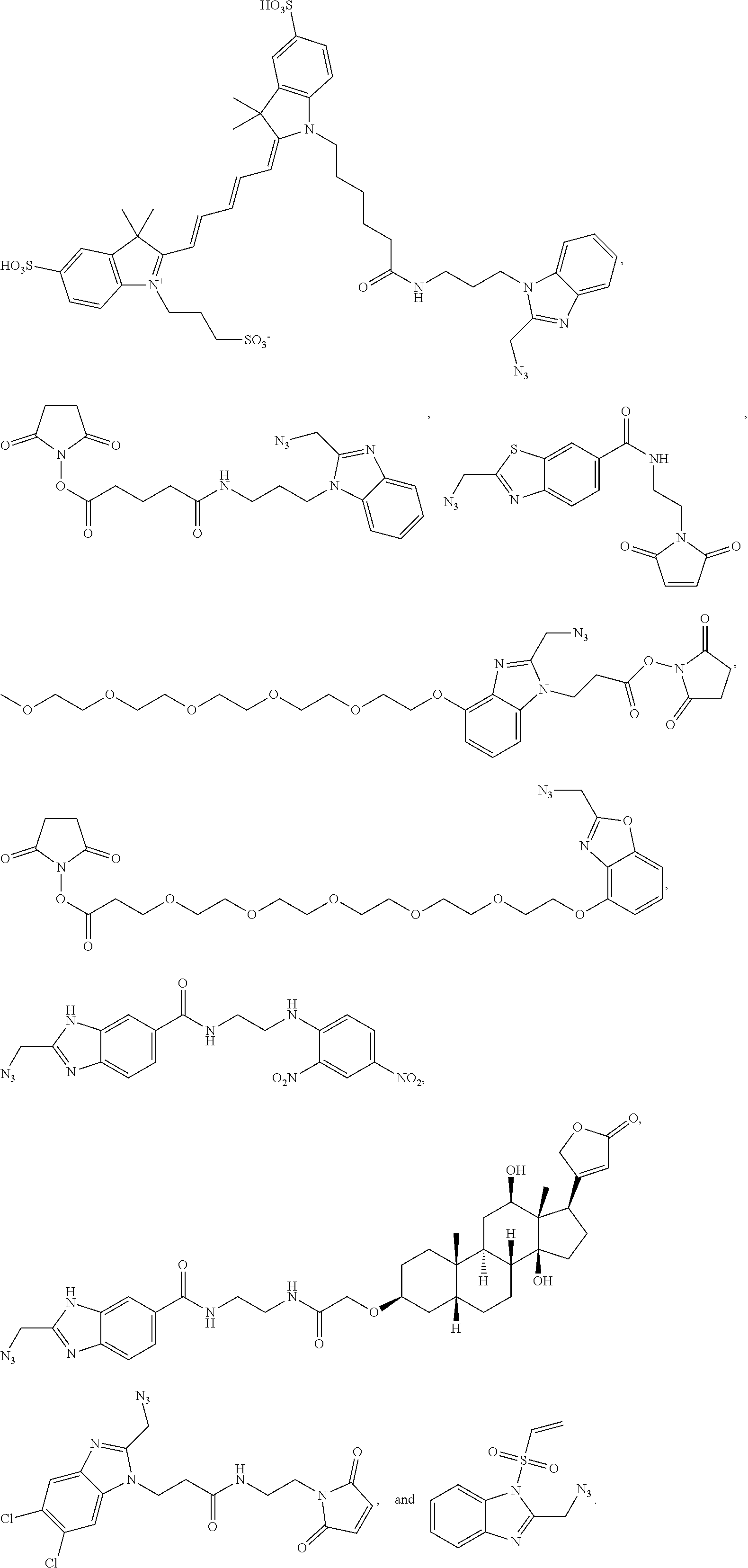

18. The compound of claim 1, wherein the compound of Formula A is represented by Formula III: 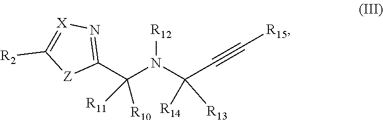
wherein
X is CR₁ or N;
Z is O, S, N—R₈, P—R₈, O═P—R₈, or O═P—OR₈;
each of R₁ and R₂ is independently selected from H, halogen, FG, BDM, L-FG, L-BDM, L, L-BS, FD, L-FD, carboxy, sulfonate, PEG, WSG, alkoxy, aryloxy, thiol, alkylthiol, arylthiol, nitro, nitroso, cyano, amino, hydroxy, phosphonyl, sulfonyl, carbonyl, boronic acid, aryl, heteroaryl, and optionally substituted versions thereof;
or R₁ and R₂ are cyclically linked to form an optionally substituted aryl or heteroaryl;
each of R₈, R₁₀, R₁₁, R₁₂, R₁₃, R₁₄, and R₁₅ is selected from independently H, alkyl, FG, BDM, L-FG, L-BDM, L, L-BS, FD, L-FD, acyl, sulfonyl, aryl, heteroaryl, and optionally substituted versions thereof.

19. The compound of claim 18, wherein X is CR₁;
Z is O, S, or N—R₈;
R₁ and R₂ are cyclically linked to form an optionally substituted aryl or heteroaryl;
each of R₈, R₁₀, and R₁₁ is independently selected from H, optionally substituted alkyl, optionally substituted aryl, and optionally substituted heteroaryl.

20. The compound of claim 18, wherein X is N;
Z is O, S, or N—R₈;
R₂ is H, halogen, carboxy, sulfonate, a PEG, alkoxy, aryloxy, aryl, or heteroaryl;
each of R₈, R₁₀, R₁₁, R₁₂, R₁₃, R₁₄, and R₁₅ is independently selected from H, or an optionally substituted alkyl, aryl, and heteroaryl.

21. The compound of claim 18, wherein X is N;
Z is O, S, or N—R₈;
R₂ is H, halogen, carboxy, sulfonate, a PEG, alkoxy, aryloxy, aryl, or heteroaryl;
each of R₈, R₁₀, R₁₁, R₁₂, R₁₃ R₁₄, and R₁₅ is independently selected from H, optionally substituted alkyl, optionally substituted aryl, and optionally substituted heteroaryl;
R₁₂ is acyl or sulfonyl.

22. The compound of claim 18, wherein R₁₅ is H, CH₃,

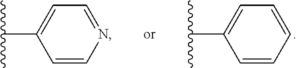 [SMILES: *c1ccncc1 ; *c1ccccc1]

23. The compound of claim 18, wherein WSG is sulfonate, PEG, carboxyalkyl, sulfonylalkyl, phosphonylalkyl, hydroxyalkyl, aminoalkyl, or tetraalkylammonium.

24. The compound of claim 18, wherein FG is an activated ester, aldehyde, maleimide, amino, hydroxylamine, or hydrazine.

25. The compound of claim 18, wherein one of R₁, R₂, R₈, R₁₀, R₁₁, R₁₂, R₁₃, R₁₄, and R₁₅ comprises a FD or L-FD.

26. The compound of claim 18, wherein BS is an antibody, antigen, protein, peptide, oligonucleotide, DNA, RNA, PNA, aptamer, or cell.

27. The compound of claim 18, wherein the compound is selected from

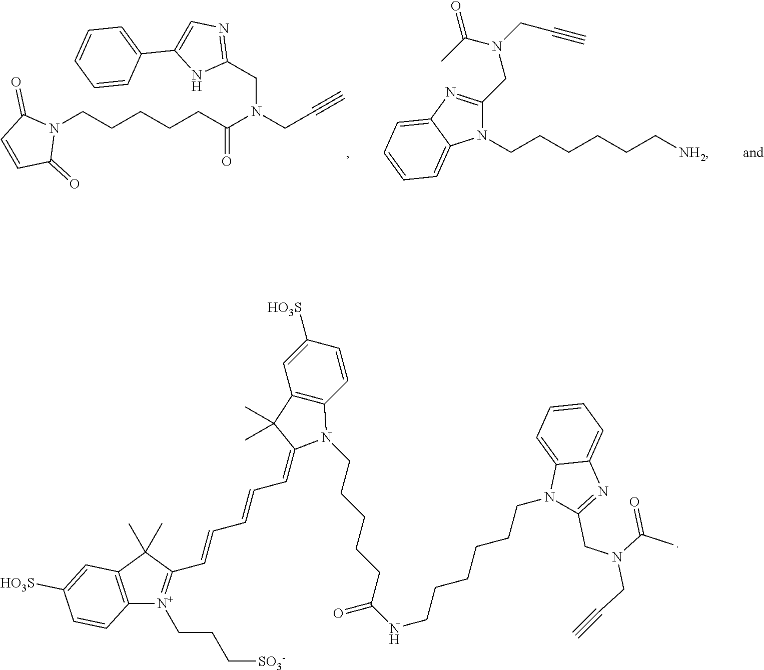

28. The compound of claim 1, wherein the compound is of Formula IV: 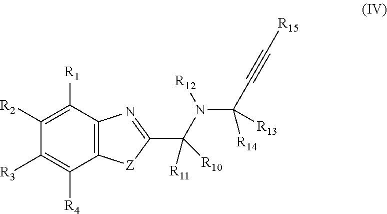
wherein
Z is O, S, N—R₈, P—R₈, O═P—R₈, or O═P—OR₈;
each of R₁, R₂, R₃, and R₄ is independently selected from H, halogen, FG, BDM, L-FG, L-BDM, L, L-BS, FD, L-FD, carboxy, sulfonate, PEG, WSG, alkoxy, aryloxy, thiol, alkylthiol, arylthiol, nitro, nitroso, cyano, amino, hydroxy, phosphonyl, sulfonyl, carbonyl, boronic acid, aryl, heteroaryl, and optionally substituted versions thereof;
each of R₈, R₁₀, R₁₁, R₁₂, R₁₃, R₁₄, R₁₅ is independently selected from H, alkyl, FG, BDM, L-FG, L-BDM, L, L-BS, FD, L-FD, acyl, sulfonyl, aryl, heteroaryl, and optionally substituted versions thereof.

29. The compound of claim 28, wherein
Z is O, S, or N—R₈;
each of R₁, R₂, R₃, and R₄ is independently selected from H, halogen, carboxy, sulfonate, PEG, alkoxy, aryloxy, aryl, heteroaryl, and optionally substituted versions thereof;
each of R₈, R₁₀, R₁₁, R₁₂, R₁₃, R₁₄, and R₁₅ is independently selected from H, optionally substituted alkyl, optionally substituted aryl, and optionally substituted heteroaryl.

30. The compound of claim 28, wherein
Z is O, S, or N—R₈;
R₂ is H, halogen, carboxy, sulfonate, PEG, alkoxy, aryloxy, aryl, heteroaryl, and optionally substituted versions thereof;
each of R₈, R₁₀, R₁₁, R₁₃, R₁₄ and R₁₅ is independently selected from H, optionally substituted alkyl, optionally substituted aryl, and optionally substituted heteroaryl;
R₁₂ is an acyl or sulfonyl.

31. The compound of claim 28, wherein R₁₅ is H, CH₃,

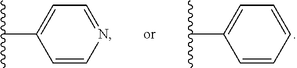 [SMILES: *c1ccncc1 ; *c1ccccc1]

32. The compound of claim 28, wherein WSG is a sulfonate, PEG, carboxyalkyl, sulfonylalkyl, phosphonylalkyl, hydroxyalkyl, aminoalkyl, or trialkylammonium.

33. The compound of claim 28, wherein FG is selected from an activated ester, aldehyde, maleimide, amino, hydroxylamine, and hydrazine.

34. The compound of claim 28, wherein one of R₁ to R₁₅ comprises FD or L-FD.

35. The compound of claim 28, wherein each of R₁, R₂, R₃, R₄, and R₈ is independently selected from H, halogen,

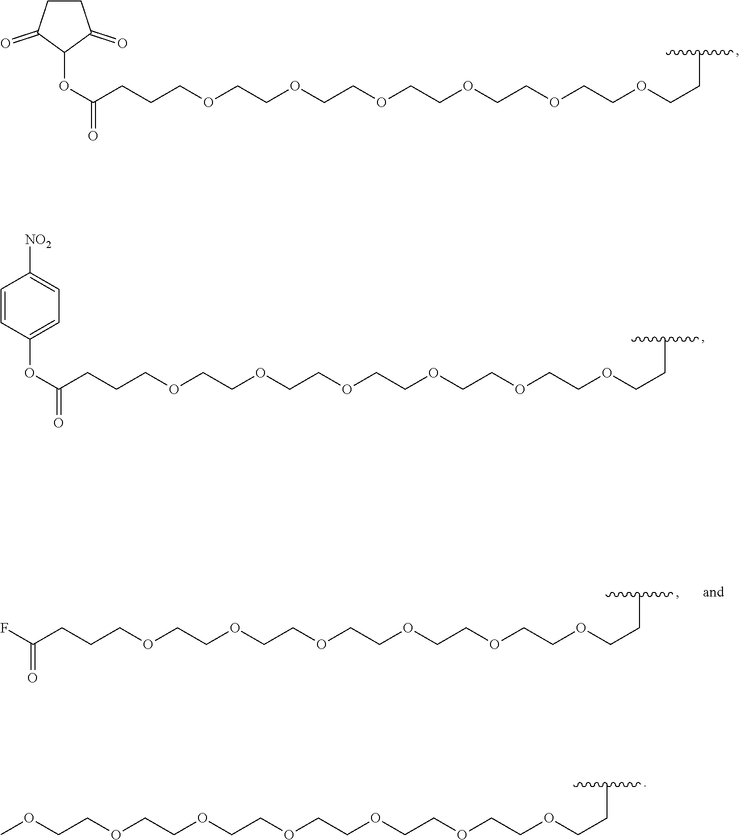 [SMILES: *CCOCCOCCOCCOCCOCCOCCCC(=O)OC1C(=O)CCC1=O ; *CCOCCOCCOCCOCCOCCOCCCC(=O)Oc1ccc([N+](=O)[O-])cc1 ; *CCOCCOCCOCCOCCOCCOCCCC(=O)F ; *CCOCCOCCOCCOCCOCCOCCOC]

36. The compound of claim 28, wherein R₁₂ is independently selected from

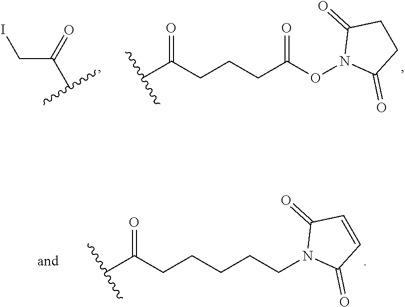 [SMILES: *C(=O)CI ; *C(=O)CCCC(=O)ON1C(=O)CCC1=O ; *C(=O)CCCCCN1C(=O)C=CC1=O]

37. The compound of claim 28, wherein BS is an antibody, an antigen, a protein, a peptide, an oligonucleotide, a DNA, an RNA, a PNA, an aptamer or a cell.

38. The compound of claim 28, wherein the compound is selected from

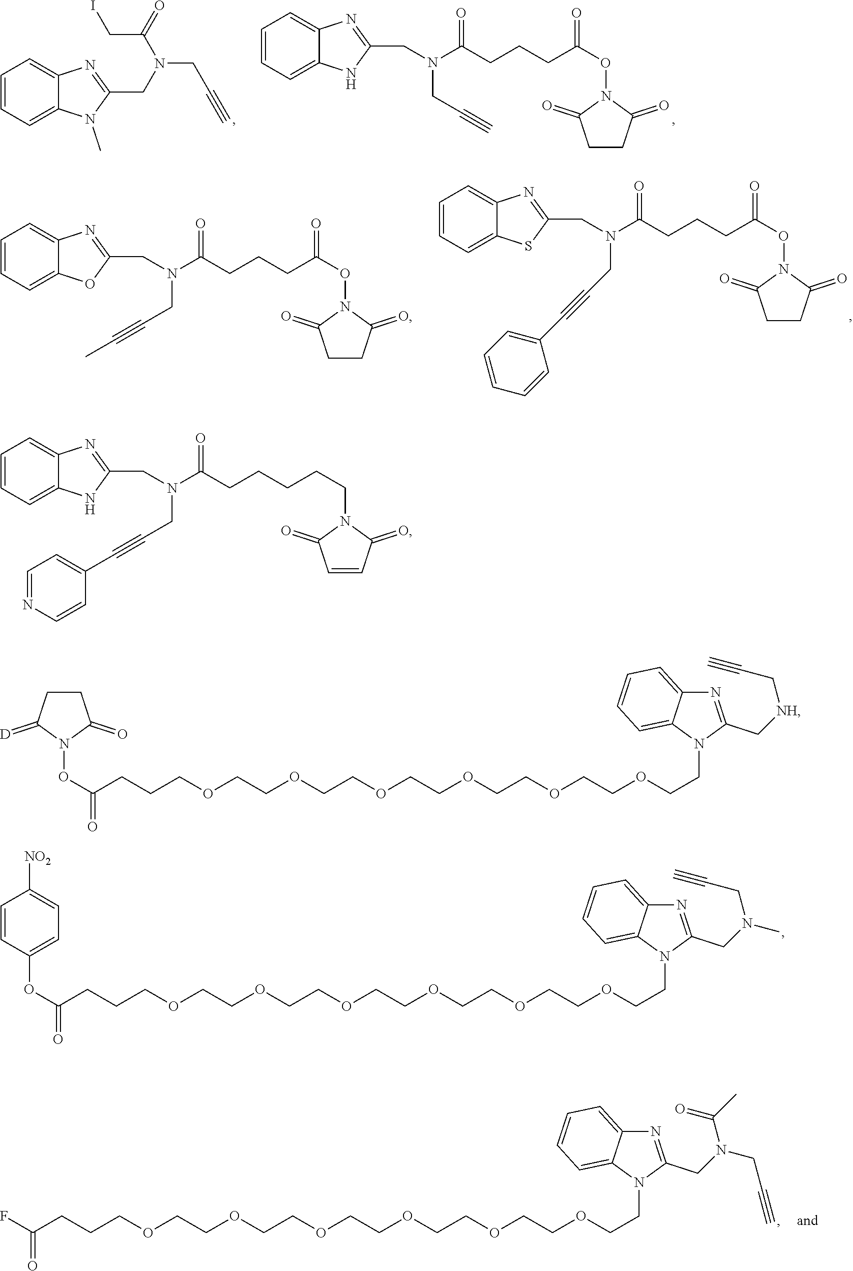

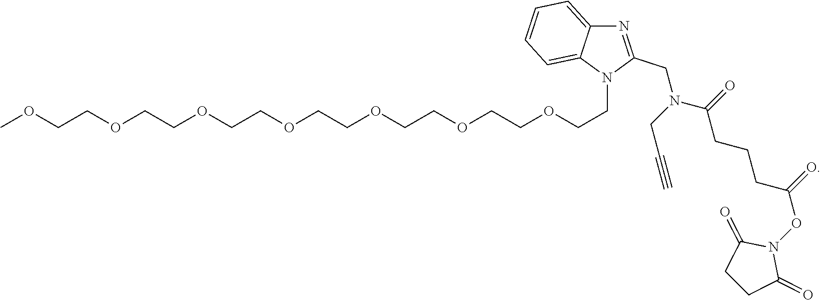 [SMILES: C#CCN(Cc1nc2ccccc2n1CCOCCOCCOCCOCCOCCOCCOC)C(=O)CCCC(=O)ON1C(=O)CCC1=O]

39. A method of preparing an azide-alkyne conjugate, comprising
a) contacting an alkyne-containing substance with an azide-containing compound according to claim 1 under conditions under which said alkyne-containing substance and the azide-containing compound react with each other to produce a triazole-containing conjugate;
b) isolating or purifying the conjugate, or detecting the conjugate in situ (e.g., by fluorescence).

40. The method of claim 39, wherein the alkyne-containing substance is selected from alkyne-modified antibody, alkyne-modified avidin, alkyne-modified streptavidin, alkyne-modified neutravidin, and alkyne-modified enzyme.

41. The method of claim 39, wherein the alkyne-containing substance is an alkyne-labeled cell or tissue.

42. A method of preparing an azide-alkyne conjugate, comprising
a) contacting an azido-containing substance with an alkyne-containing compound according to claim 1 under conditions under which said azido-containing substance and alkyne-containing compound react with each other to produce a triazole-containing conjugate;
b) isolating or purifying the conjugate, or detecting the conjugate in situ (e.g., by fluorescence).

43. The method of claim 42, wherein the azido-containing substance is selected from an azido-modified antibody, azido-modified avidin, azido-modified streptavidin, azido-modified neutravidin, and azido-modified enzyme.

44. The method of claim 42, wherein the azido-containing substance is an azido-labeled cell or tissue.

45. A method of preparing a conjugate, comprising
a) contacting a substance (e.g., moiety of interest) with an azide-containing compound of Formula I or II that comprises a linked FG under conditions under which said substance and the FG of said compound react to produce an azide-modified substance;
b) incubating the said azide-modified substance with an alkyne-containing substance to produce a conjugate;
c) isolating or purifying the conjugate, or detecting the conjugate (e.g., in situ by fluorescence).

46. A method of preparing a conjugate, comprising the steps of:
a) contacting a substance (e.g., moiety of interest) with an alkyne-containing compound of Formula III or IV that comprises a linked FG under conditions under which said substance and said compound react to produce an azide-modified substance;
b) incubating the azide-modified substance of a) with an azido-containing substance to produce a conjugate;
c) isolating or purifying the conjugate, or detecting the conjugate (e.g., in situ by fluorescence).

47. A method of detecting an analyte in a sample, comprising the steps of:
a) contacting said sample comprising an analyte with a detection reagent comprising a compound of Formula I, II, III or IV, or a conjugate thereof, under conditions under which said detection reagent will react with, or specifically bind said analyte; and
b) detecting the detection reagent bound analyte (e.g., by fluorescence).
